(19) United States
(12) Patent Application Publication
(10) Pub. No.: US 2025/0073471 A1
(43) Pub. Date: Mar. 6, 2025

(54) SYSTEM AND METHODS FOR AUTOMATED DEEP BRAIN STIMULATION PARAMETER SELECTION VIA META-ACTIVE LEARNING OF EVOKED POTENTIALS

(71) Applicants: Georgia Tech Research Corporation, Atlanta, GA (US); Emory University, Atlanta, GA (US)

(72) Inventors: Eric Richard Cole, Atlanta, GA (US); Matthew C. Gombolay, Atlanta, GA (US); Robert Gross, Atlanta, GA (US); Svjetlana Miocinovic, Atlanta, GA (US); Enrico Opri, Atlanta, GA (US); Mariah Schrum, Atlanta, GA (US)

(21) Appl. No.: 18/819,710

(22) Filed: Aug. 29, 2024

Related U.S. Application Data
(60) Provisional application No. 63/535,144, filed on Aug. 29, 2023.

(51) Int. Cl. A61N 1/36

(52) U.S. Cl. CPC A61N 1/36146; A61N 1/36067; A61N 1/36139

(57) ABSTRACT

An exemplary embodiment of the present disclosure provides a system for generating a set of stimulation parameters, the system comprising at least one processor and a memory in communication with the at least one processor and having stored thereon instructions that, when executed by the at least one processor, is configured to cause the system to analyze, using a machine learning model, data from the deep brain stimulation device or an electromyography and generate, in response to analyzing, at least in part, the data, a set of stimulation parameters for deep brain stimulation.

### CROSS-REFERENCE TO RELATED APPLICATIONS

This application claims priority to U.S. Provisional Patent Application No. 63/535,144, filed 29 Aug. 2023, which is hereby incorporated by reference herein in its entirety as if fully set forth below.

### GOVERNMENT LICENSE RIGHTS

This invention was made with government support under NS125143, awarded by the National Institutes of Health. The government has certain rights in the invention.

### FIELD OF THE DISCLOSURE

The various embodiments of the present disclosure relate generally to systems and methods for generating a set of stimulation parameters, and more particularly to using algorithms and models with data from a deep brain stimulation device to generate a set of parameters.

### GOVERNMENT LICENSE RIGHTS

This invention was made with government support under Agreement No. NINDS 5R01NS125143, awarded by the National Institute of Neurological Disorders and Stroke. The government has certain rights in the invention.

### BACKGROUND

Programming a deep brain stimulation device (DBS) for the treatment of Parkinson's disease can have challenges. For example, finding the best selection of parameters for the device when they differ for each patient and with 100,000's of available parameter combinations is difficult. Providing a clinician a patient-specific list of best parameters for faster programming of the device is essential for the treatment of the disease. Conventional methods use a trial-and-error search through the various parameter combinations and is typically conducted by the clinician over multiple patient visits. Using this approach can take months to find the best therapy for a patient. To give patients their best therapy more quickly and to free up neurologists' time for the dedication to other matters, a solution is needed. A more efficient method can grant patients better access and outcomes. A solution to the selection of parameters for the device can also be a long-term solution for the treatment of other diseases, such as epilepsy, other movement disorders, and neuropsychiatric DBS for example.

What is needed, therefore, is a system that uses a hybrid meta-learning and mathematical programming approach that can enable efficient, safe, and computationally fast optimization of a latent robotic system to select the best parameters for a deep brain stimulation device. Embodiments of the present disclosure address the above concerns as well as other needs that will become apparent upon reading the description below in conjunction with the drawings.

### BRIEF SUMMARY

An exemplary embodiment of the present disclosure provides a system comprising at least one processor and a memory in communication with the at least one processor and having stored thereon instructions that, when executed by the at least one processor, is configured to cause the system to analyze, using a machine learning model, data from the deep brain stimulation device or an electromyography, and generate, in response to analyzing, at least in part, the data, a set of stimulation parameters for deep brain stimulation.

In any of the embodiments disclosed herein, at least a portion of the data can comprise, at least in part, sample data from a neural network or biomarker data collected from a population of patients.

In any of the embodiments disclosed herein, the biomarker data collected from the population of patients can be recorded by the deep brain stimulation device or the electromyography.

In any of the embodiments disclosed herein, the set of stimulation parameters can be generated using the machine learning model trained to select parameters using historical data of previous sets of stimulation parameters.

In any of the embodiments disclosed herein, the machine learning model can be configured to maximize deep brain stimulation local evoked potentials (DLEP) and minimize EMG-measured motor evoked potentials (mEP).

In any of the embodiments disclosed herein, the machine learning model can be configured to predict biomarker values for unseen parameters by programming deep brain stimulation parameters in a simulation environment.

In any of the embodiments disclosed herein, the instructions, when executed by the at least one processor, can be configured to cause the system to instruct the deep brain stimulation device to deliver electromagnetic energy to at least a portion of a brain of a user, wherein one or more characteristics of the electromagnetic energy is based, at least in part, on the set of stimulation parameters.

In any of the embodiments disclosed herein, the memory, when executed by the at least one processor can be further configured to cause the system to determine whether at least a portion of the set of stimulation parameters fall within a predetermined safety threshold.

In any of the embodiments disclosed herein, the machine learning model can be configured to predict a probability of returning to a safe state, take an action outside of the safe state to gain information, and return to the safe state within a preset time period.

An exemplary embodiment of the present disclosure provides a method for selecting parameters for a deep brain stimulation device comprising analyzing, using a machine learning model, data from the deep brain stimulation device or an electromyography and generating, in response to analyzing, at least in part, the data, a set of stimulation parameters for deep brain stimulation.

In any of the embodiments disclosed herein, the data can comprise, at least in part, sample data from a neural network or biomarker data collected from a population of patients.

In any of the embodiments disclosed herein, the set of stimulation parameters can be generated using the machine learning model trained to select parameters using historical data of previous sets of stimulation parameters.

In any of the embodiments disclosed herein, the method can further include instructing the deep brain stimulation device to deliver electromagnetic energy to at least a portion of a brain of a user, wherein one or more characteristics of the electromagnetic energy is based, at least in part, on the set of stimulation parameters.

In any of the embodiments disclosed herein, the machine learning model can be configured to predict biomarker values for unseen parameters by programming deep brain stimulation parameters in a simulation environment.

In any of the embodiments disclosed herein, the method can further comprise predicting, using the machine learning model, a probability of returning to a safe state, taking an action outside of the safe state to gain information, and returning to the safe state within a preset time period.

An exemplary embodiment of the present disclosure provides a non-transitory computer readable medium having stored thereon instructions comprising executable code which when executed by one or more processors, causes the one or more processors to analyze, using a machine learning model, data from a deep brain stimulation device or an electromyography, and generate, in response to analyzing, at least in part, the data, a set of stimulation parameters for deep brain stimulation.

In any of the embodiments disclosed herein, the data can comprise, at least in part, sample data from a neural network or biomarker data collected from a population of patients.

In any of the embodiments disclosed herein, the machine learning model can be configured to select the set of stimulation parameters by testing parameters in a simulation environment using historical data of previous sets of stimulation parameters.

In any of the embodiments disclosed herein, the machine learning model can be configured to predict biomarker values for unseen parameters while maximizing deep brain stimulation local evoked potentials (DLEP) and minimizing EMG-measured motor evoked potentials (mEP).

In any of the embodiments disclosed herein, the instructions can further comprise executable code which when executed by one or more processors, causes the one or more processors to predict, using the machine learning model, a probability of returning to a safe state, take an action outside of the safe state to gain information; and return to the safe state within a preset time period.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a system in accordance with an exemplary embodiment of the present invention.

FIG. 2 provides a flowchart of a meta-active learning architecture, in accordance with an exemplary embodiment of the present invention. The learned brain model can sample new patients and learn a policy that results in the best stimulation for a given patient. A prior embedding allows for learning the action that results in the best biomarkers. The flowchart is a standard ReLU model that should provide superior performance and efficiency by leveraging population level data for a search.

FIG. 3 provides an image of brain models, in accordance with an exemplary embodiment of the present invention. A can represent the parameters, w can represent a patient embedding, and s can represent biomarkers.

FIG. 4 provides a meta-learning framework, grounded in the UAV application. The red, blue and green curves can represent the hypothetical manifolds within the distribution of damage scenarios. The meta-learning algorithm samples from the distribution of damage conditions and learns a function describing the expected informativeness of taking an action. The present disclosure can embed this in a MILP to enforce safety-constraints, thereby ensuring safe flight while enabling efficient record from damage.

FIG. 5 provides an image in accordance with an exemplary embodiment of the present invention. This figure depicts a g volume of safety, i.e., convex constraints around reference trajectory \vec{s}ᵣ(t). Action, \vec{a}^{(t)}, is an exploratory action, which may bring the system outside of the safe region. Given \hat{ƒ}_{ψ}_{^{(t)}}, Safe MetAL ensures the probability that \vec{a}^{(t+2)} returns the system to a safe state is at least 1−ϵ.

FIGS. 6A-C provides empirical validation, generated via Monte Carlo simulation, in the robotic UAV domain benchmarking algorithm accuracy per time step (FIG. 6A), overall expected informativeness over the time horizon (FIG. 6B), and vs. computation time (FIG. 6C). Error is calculated in batches of three-time steps, enabling the robot to deviate from the safe region temporarily to gain information. The results shown in FIG. 6A comply with safety results reported in FIG. 6B.

FIGS. 7A-C provides the empirical validation in the DBS domain, benchmarking algorithm accuracy per time step (FIG. 7A), overall (FIG. 7B), and vs. computation time (FIG. 7C). The optimal parameter accuracy is defined as

$$1 - \frac{{a}^{\to *} - {a}^{\hat{\to }}}{{a}^{\to *}} .$$

where \vec{a}* is the optimal stimulation parameter and \vec{â}: can be the predicted parameter. In FIG. 7B, the present disclosure also can report the ground truth safety of the algorithm compared to baselines. The results showing in FIG. 7A can comply with the safety results reported in FIG. 7B.

FIGS. 8A-C provides an ablation analysis and the trade-off between expected informativeness and safety. In FIGS. 8A (UAV domain) and 8B (DBS domain), the present disclosure can set λ=0, meaning there is no active learning and only safety is maximized. In both domains, the meta-learned acquisition function can be an important component to achieve efficient learning. FIG. 8C, can show an ablation study, demonstrating the trade-off between expected informativeness and safety in the UAV domain when the present disclosure varies 2. The present disclosure can show that tune A to achieve the desired tradeoff between expected informativeness and safe operation.

FIGS. 9A and 9B provides images of DBS parameters in accordance with an exemplary embodiment of the present invention.

FIGS. 10A and 10B provides a chart (FIG. 10A) and a system (FIG. 10B) in accordance with an exemplary embodiment of the present invention.

FIG. 11 provides exemplary DBS devices in accordance with an exemplary embodiment of the present invention.

FIG. 12 provides an image in accordance with an exemplary embodiment of the present invention.

FIG. 13 provides an image in accordance with an exemplary embodiment of the present invention.

FIG. 14 provides an image of LFP recording from adjacent DBS contacts in accordance with an exemplary embodiment of the present invention.

FIG. 15 provides an image of EMG on facial and forearm muscles in accordance with an exemplary embodiment of the present invention.

FIG. 16 provides a chart of side effect prediction accuracy in accordance with an exemplary embodiment of the present invention.

FIG. 17 provides charts of detection examples in accordance with an exemplary embodiment of the present invention.

FIG. 18 provides a chart of detection accuracy vs. muscle type in accordance with an exemplary embodiment of the present invention.

FIG. 19 provides an example of machine learning for DBS in accordance with an exemplary embodiment of the present invention.

FIGS. 20A and 20B provides charts of patient examples in accordance with an exemplary embodiment of the present invention.

FIG. 21 provides a chart of meta learning in accordance with an exemplary embodiment of the present invention.

FIG. 22A-B provides flows of active learning in accordance with an exemplary embodiment of the present invention.

FIG. 23 provides a chart of learned embedding of 52 intraoperative patient responses in accordance with an exemplary embodiment of the present invention.

FIGS. 24A-C provides a chart (FIG. 24A) and flows (FIGS. 24B-C), in accordance with an exemplary embodiment of the present invention.

FIG. 25 provides flows in accordance with an exemplary embodiment of the present invention.

FIG. 26 provides charts in accordance with an exemplary embodiment of the present invention.

FIG. 27 provides a chart in accordance with an exemplary embodiment of the present invention.

FIG. 28 provides a flow of a closed-loop intraoperative parameter selection in accordance with an exemplary embodiment of the present invention.

FIG. 29 provides a chart of MEP latencies depending on muscle types in accordance with an exemplary embodiment of the present invention.

FIGS. 30A-B provides charts of subclinical prediction of side-effect amplitude thresholds in accordance with an exemplary embodiment of the present invention.

FIG. 31 provides a chart of motor EP delays depending on muscle type in accordance with an exemplary embodiment of the present invention.

FIG. 32 provides a chart of grouping leads by ACPC coordinates in accordance with an exemplary embodiment of the present invention.

FIG. 33 provides a chart of grouping leads by LeadDBS distances in accordance with an exemplary embodiment of the present invention.

FIG. 34 provides a chart of grouping leads by LeadDBS distances in accordance with an exemplary embodiment of the present invention.

FIG. 35 provides charts of permutation-based cluster analysis in accordance with an exemplary embodiment of the present invention. The top row of clusters can depict a silhouette score and the bottom row of clusters can depict a non-contiguous clustering coefficient.

FIGS. 36A-F provides charts of p-values in cluster analysis in accordance with an exemplary embodiment of the present invention.

FIGS. 37A-B provides charts of meta-al that can find optimal parameters in silico in accordance with an exemplary embodiment of the present invention.

FIGS. 38A-B provides charts of meta-AL that can find optimal parameters in silico in accordance with an exemplary embodiment of the present invention.

FIGS. 39A-B provides charts of embedding that captures lead placement information in accordance with an exemplary embodiment of the present invention.

FIG. 40 provides charts of motor EP's predict DBS-induced side effects in accordance with an exemplary embodiment of the present invention.

FIGS. 41A-F provides charts of monopolar in accordance with an exemplary embodiment of the present invention.

FIGS. 42A-F provides charts of monopolar segmented in accordance with an exemplary embodiment of the present invention.

FIG. 43 provides an image of mEP-inducing parameters that can be consistent with internal capsule activation in accordance with an exemplary embodiment of the present invention.

FIG. 44 provides an image of mEP-inducing parameters that can be consistent with internal capsule activation in accordance with an exemplary embodiment of the present invention.

FIGS. 45A-B provides charts of meta-learning predictions of DBS responses in accordance with an exemplary embodiment of the present invention.

FIG. 46 provides a chart of meta-learning predictions of DBS responses in accordance with an exemplary embodiment of the present invention.

FIG. 47 provides a chart of meta-learning predictions of DBS responses in accordance with an exemplary embodiment of the present invention.

FIG. 48 provides a chart of meta-al that can find optimal parameters in silico in accordance with an exemplary embodiment of the present invention.

FIG. 49 provides a flow of a model architecture in accordance with an exemplary embodiment of the present invention.

FIG. 50 provides an image of an algorithm in accordance with an exemplary embodiment of the present invention.

FIG. 51 provides an image of an algorithm in accordance with an exemplary embodiment of the present invention.

FIG. 52 provides a flowchart in accordance with an exemplary method of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Various systems, methods, and computer-readable mediums are disclosed and will now be described.

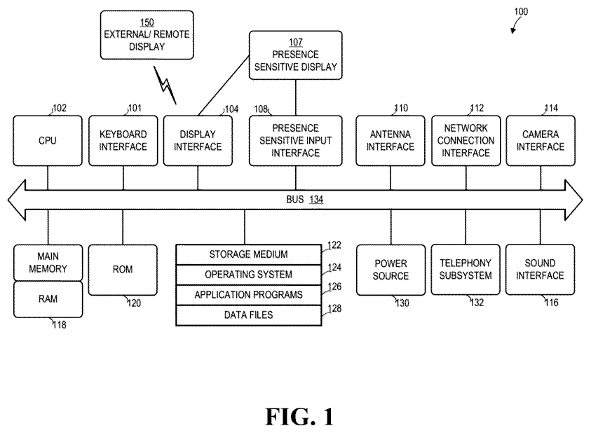

FIG. 1 depicts a block diagram of an illustrative computing device architecture 100, according to an example embodiment. Certain aspects of FIG. 1 may be embodied in a computing device 100. As desired, embodiments of the disclosed technology may include a computing device with more or less of the components illustrated in FIG. 1. It will be understood that the computing device architecture 100 is provided for example purposes only and does not limit the scope of the various embodiments of the present disclosed systems, methods, and computer-readable mediums.

The computing device architecture 100 of FIG. 1 includes a CPU 102, where computer instructions are processed; a display interface 104 that acts as a communication interface and provides functions for rendering video, graphics, images, and texts on the display. In certain embodiments of the disclosed technology, the display interface 104 may be directly connected to a local display, such as a touch-screen display associated with a mobile computing device. In another example embodiment, the display interface 104 may be configured for providing data, images, text, and other information for an external/remote display that is not necessarily physically connected to the mobile computing device. For example, a desktop monitor may be utilized for mirroring graphics and other information that is presented on a mobile computing device. In certain some embodiments, the display interface 104 may wirelessly communicate, for example, via a Wi-Fi channel or other available network connection interface 112 to the external/remote display.

In an example embodiment, the network connection interface 112 may be configured as a communication interface and may provide functions for rendering video, graphics, images, text, other information, or any combination thereof on the display. In one example, a communication interface may include a serial port, a parallel port, a general-purpose input and output (GPIO) port, a game port, a universal serial bus (USB), a micro-USB port, a high-definition multimedia (HDMI) port, a video port, an audio port, a Bluetooth port, a near-field communication (NFC) port, another like communication interface, or any combination thereof.

The computing device architecture 100 may include a keyboard interface 106 that provides a communication interface to a keyboard. In one example embodiment, the computing device architecture 100 may include a presence-sensitive display interface 107 for connecting to a presence-sensitive display. According to certain some embodiments of the disclosed technology, the presence-sensitive display interface 107 may provide a communication interface to various devices such as a pointing device, a touch screen, a depth camera, etc. which may or may not be associated with a display.

The computing device architecture 100 may be configured to use an input device via one or more of input/output interfaces (for example, the keyboard interface 106, the display interface 104, the presence sensitive display interface 107, network connection interface 112, camera interface 114, sound interface 116, etc.) to allow a user to capture information into the computing device architecture 100. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 100 or may be a separate device. For example, the input device may be an accelerometer, a magnetometer, a digital camera, a microphone, and an optical sensor.

Example embodiments of the computing device architecture 100 may include an antenna interface 110 that provides a communication interface to an antenna; a network connection interface 112 that provides a communication interface to a network. In certain embodiments, a camera interface 114 is provided that acts as a communication interface and provides functions for capturing digital images from a camera. In certain embodiments, a sound interface 116 is provided as a communication interface for converting sound into electrical signals using a microphone and for converting electrical signals into sound using a speaker. According to example embodiments, a random-access memory (RAM) 118 is provided, where computer instructions and data may be stored in a volatile memory device for processing by the CPU 102.

According to an example embodiment, the computing device architecture 100 includes a read-only memory (ROM) 120 where invariant low-level system code or data for basic system functions such as basic input and output (I/O), startup, or reception of keystrokes from a keyboard are stored in a non-volatile memory device. According to an example embodiment, the computing device architecture 100 includes a storage medium 122 or other suitable type of memory (e.g., RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 124, application programs 126 (including, for example, a web browser application, a widget or gadget engine, and or other applications, as necessary) and data files 128 are stored. According to an example embodiment, the computing device architecture 100 includes a power source 130 that provides an appropriate alternating current (AC) or direct current (DC) to power components. According to an example embodiment, the computing device architecture 100 includes a telephony subsystem 132 that allows the transmission and receipt of sound over a telephone network. The constituent devices and the CPU 102 communicate with each other over a bus 134.

According to an example embodiment, the CPU 102 has appropriate structure to be a computer processor. In one arrangement, the CPU 102 may include more than one processing unit. The RAM 118 interfaces with the computer bus 134 to provide quick RAM storage to the CPU 102 during the execution of software programs such as the operating system application programs, and device drivers. More specifically, the CPU 102 loads computer-executable process steps from the storage medium 122 or other media into a field of the RAM 118 in order to execute software programs. Data may be stored in the RAM 118, where the data may be accessed by the computer CPU 102 during execution. In one example configuration, the device architecture 100 includes at least 125 MB of RAM, and 256 MB of flash memory.

The storage medium 122 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 122, which may comprise a machine-readable storage medium.

According to one example embodiment, the term computing device, as used herein, may be a CPU, or conceptualized as a CPU (for example, the CPU 102 of FIG. 1). In this example embodiment, the computing device may be coupled, connected, and/or in communication with one or more peripheral devices, such as display. In this example embodiment, the computing device may output content to its local display and/or speaker(s). In another example embodiment, the computing device may output content to an external display device (e.g., over Wi-Fi) such as a TV or an external computing system.

In some embodiments of the disclosed technology, the computing device 100 may include any number of hardware and/or software applications that are executed to facilitate any of the operations. In some embodiments, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be utilized to receive or collect data and/or user instructions from a wide variety of input devices. Received data may be processed by one or more computer processors as desired in various embodiments of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

The computing device architecture 100 may contain programs that train, implement, store, receive, retrieve, and/or transmit one or more machine learning models. Machine learning models may include a neural network model, a generative adversarial model (GAN), a recurrent neural network (RNN) model, a deep learning model (e.g., a long short-term memory (LSTM) model), a random forest model, a convolutional neural network (CNN) model, a support vector machine (SVM) model, logistic regression, XGBoost, and/or another machine learning model. Models may include an ensemble model (e.g., a model comprised of a plurality of models). In some embodiments, training of a model may terminate when a training criterion is satisfied. Training criterion may include a number of epochs, a training time, a performance metric (e.g., an estimate of accuracy in reproducing test data), or the like. The computing device architecture 100 may be configured to adjust model parameters during training. Model parameters may include weights, coefficients, offsets, or the like. Training may be supervised or unsupervised.

The computing device architecture 100 may be configured to train machine learning models by optimizing model parameters and/or hyperparameters (hyperparameter tuning) using an optimization technique, consistent with disclosed embodiments. Hyperparameters may include training hyperparameters, which may affect how training of the model occurs, or architectural hyperparameters, which may affect the structure of the model. An optimization technique may include a grid search, a random search, a gaussian process, a Bayesian process, a Covariance Matrix Adaptation Evolution Strategy (CMA-ES), a derivative-based search, a stochastic hill-climb, a neighborhood search, an adaptive random search, or the like. The computing device architecture 100 may be configured to optimize statistical models using known optimization techniques.

Furthermore, the computing device architecture 100 may include programs configured to retrieve, store, and/or analyze properties of data models and datasets. For example, computing device architecture 100 may include or be configured to implement one or more data-profiling models. A data-profiling model may include machine learning models and statistical models to determine the data schema and/or a statistical profile of a dataset (e.g., to profile a dataset), consistent with disclosed embodiments. A data-profiling model may include an RNN model, a CNN model, or other machine-learning model.

The computing device architecture 100 may include algorithms to determine a data type, key-value pairs, row-column data structure, statistical distributions of information such as keys or values, or other property of a data schema may be configured to return a statistical profile of a dataset (e.g., using a data-profiling model). The computing device architecture 100 may be configured to implement univariate and multivariate statistical methods. The computing device architecture 100 may include a regression model, a Bayesian model, a statistical model, a linear discriminant analysis model, or other classification model configured to determine one or more descriptive metrics of a dataset. For example, computing device architecture 100 may include algorithms to determine an average, a mean, a standard deviation, a quantile, a quartile, a probability distribution function, a range, a moment, a variance, a covariance, a covariance matrix, a dimension and/or dimensional relationship (e.g., as produced by dimensional analysis such as length, time, mass, etc.) or any other descriptive metric of a dataset.

The computing device architecture 100 may be configured to return a statistical profile of a dataset (e.g., using a data-profiling model or other model). A statistical profile may include a plurality of descriptive metrics. For example, the statistical profile may include an average, a mean, a standard deviation, a range, a moment, a variance, a covariance, a covariance matrix, a similarity metric, or any other statistical metric of the selected dataset. In some embodiments, computing device architecture 100 may be configured to generate a similarity metric representing a measure of similarity between data in a dataset. A similarity metric may be based on a correlation, covariance matrix, a variance, a frequency of overlapping values, or other measure of statistical similarity.

The computing device architecture 100 may be configured to generate a similarity metric based on data model output, including data model output representing a property of the data model. For example, computing device architecture 100 may be configured to generate a similarity metric based on activation function values, embedding layer structure and/or outputs, convolution results, entropy, loss functions, model training data, or other data model output). For example, a synthetic data model may produce first data model output based on a first dataset and a produce data model output based on a second dataset, and a similarity metric may be based on a measure of similarity between the first data model output and the second-data model output. In some embodiments, the similarity metric may be based on a correlation, a covariance, a mean, a regression result, or other similarity between a first data model output and a second data model output. Data model output may include any data model output as described herein or any other data model output (e.g., activation function values, entropy, loss functions, model training data, or other data model output). In some embodiments, the similarity metric may be based on data model output from a subset of model layers. For example, the similarity metric may be based on data model output from a model layer after model input layers or after model embedding layers. As another example, the similarity metric may be based on data model output from the last layer or layers of a model.

The computing device architecture 100 may be configured to classify a dataset. Classifying a dataset may include determining whether a dataset is related to another datasets. Classifying a dataset may include clustering datasets and generating information indicating whether a dataset belongs to a cluster of datasets. In some embodiments, classifying a dataset may include generating data describing the dataset (e.g., a dataset index), including metadata, an indicator of whether data element includes actual data and/or synthetic data, a data schema, a statistical profile, a relationship between the test dataset and one or more reference datasets (e.g., node and edge data), and/or other descriptive information. Edge data may be based on a similarity metric. Edge data may and indicate a similarity between datasets and/or a hierarchical relationship (e.g., a data lineage, a parent-child relationship). In some embodiments, classifying a dataset may include generating graphical data, such as anode diagram, a tree diagram, or a vector diagram of datasets. Classifying a dataset may include estimating a likelihood that a dataset relates to another dataset, the likelihood being based on the similarity metric.

The computing device architecture 100 may include one or more data classification models to classify datasets based on the data schema, statistical profile, and/or edges. A data classification model may include a convolutional neural network, a random forest model, a recurrent neural network model, a support vector machine model, or another machine learning model. A data classification model may be configured to classify data elements as actual data, synthetic data, related data, or any other data category. In some embodiments, computing device architecture 100 is configured to generate and/or train a classification model to classify a dataset, consistent with disclosed embodiments.

The computing device architecture 100 may also contain one or more prediction models. Prediction models may include statistical algorithms that are used to determine the probability of an outcome, given a set amount of input data. For example, prediction models may include regression models that estimate the relationships among input and output variables. Prediction models may also sort elements of a dataset using one or more classifiers to determine the probability of a specific outcome. Prediction models may be parametric, non-parametric, and/or semi-parametric models.

In some examples, prediction models may cluster points of data in functional groups such as “random forests.” Random Forests may comprise combinations of decision tree predictors. (Decision trees may comprise a data structure mapping observations about something, in the “branch” of the tree, to conclusions about that thing's target value, in the “leaves” of the tree.) Each tree may depend on the values of a random vector sampled independently and with the same distribution for all trees in the forest. Prediction models may also include artificial neural networks. Artificial neural networks may model input/output relationships of variables and parameters by generating a number of interconnected nodes which contain an activation function. The activation function of a node may define a resulting output of that node given an argument or a set of arguments. Artificial neural networks may generate patterns to the network via an ‘input layer’, which communicates to one or more “hidden layers” where the system determines regressions via a weighted connections. Prediction models may additionally or alternatively include classification and regression trees, or other types of models known to those skilled in the art. To generate prediction models, the computing device architecture may analyze information applying machine-learning methods.

The computing device architecture 100 may include programs (scripts, functions, algorithms) to configure data for visualizations and provide visualizations of datasets and data models on the user device. This may include programs to generate graphs and display graphs. The computing device architecture 100 may include programs to generate histograms, scatter plots, time series, or the like on the user device. The computing device architecture 100 may also be configured to display properties of data models and data model training results including, for example, architecture, loss functions, cross entropy, activation function values, embedding layer structure and/or outputs, convolution results, node outputs, or the like on the user device.

### AI Selection of Deep Brain Stimulation Parameters

Robots can have the ability to safely and efficiently learn to operate in new environments, as it is difficult for engineers to explicitly program responses for multiple contingency. Robotic vehicles that could safely and efficiently learn their own dynamics would be capable of adapting to novel damage without crashing or having to halt operation. In healthcare, robotic devices, such as deep brain stimulation (DBS) for epilepsy therapy, could automatically learn an optimal waveforms to reduce harmful electrical activity in the brain without patient-specific, manual tuning by a physician. Active learning techniques seek to address this problem by utilizing an acquisition function to predict the expected informativeness of a data point, which is defined as the change in model's testing accuracy when adding a new data point to the training set. By accurately estimating expected informativeness of a data point, data points can be judiciously selected to improve model accuracy and reduce uncertainty.

Researchers have previously investigated active learning techniques for sample efficient learning. However, prior work in active learning can suffer from three weaknesses: 1) an inability to accurately quantify expected informativeness, 2) a lack of generalizability, and 3) a lack of safety considerations. Active learning approaches typically can hand-engineer heuristics or acquisition functions to select a best action. However, these heuristics can be proxies for true informativeness of a data point and may not accurately quantify the actual informativeness of a data point when updating the model with this new training data. Additionally, heuristics that are well suited for one active learning domain may not be effective in another. The few meta-active learning approaches proposed in recent years can rely on hand-engineered features which reduce generalizability and can need expert feature selection. Furthermore, prior approaches do not consider applications in safety critical domains in which constraints can be placed on the acquisition function to prevent the model from sampling unsafe configurations.

Yet, efficient learning is not the only criteria that can be met when dealing with safety critical domains. For example, when learning the model of a damaged UAV, one can reason about the safety of the system 100 in addition to expected informativeness of an action to prevent the UAV from entering into an unrecoverable configuration. If the dynamics model of the damaged UAV can be learned efficiently and safely, the UAV may be able to safely land or even complete its assigned task despite the damage.

A key to the approach is to safely and efficiently meta-learn an acquisition function based on a learned representation of sample history that can accurately quantify the expected informativeness for an unknown, latent model when taking a given action and experiencing the resultant state. By directly encoding this acquisition function in a chance-constrained mixed-integer linear program (MILP), the present disclosure can simultaneously enforce safety guarantees while taking an action which can maximizes expected informativeness. This acquisition function is meta-learned offline over a distribution of tasks, which can allow the policy to benefit from past experience and can provide a more robust measure of the value of a labeled data point.

This method can include (1) characterization and automated detection of evoked potential biomarkers; (2) developing meta-active learning in silico; and (3) building a real-time Python system 100 for intraoperative DBS programming. For neural biomarkers of pathway activation, the goal of DBS programming is to maximize symptom relief (STN) and avoid side effects (internal capsule). The present disclosure can have neural biomarkers of pathway activation. MEP's predict DBS-induced side effects included 10 patients, 319 total stimulation settings and 2-8 recorded muscles per patient. Meta-Learning for simulating DBS programming can include 52 patients, 1374 total stimulation settings (10-40 per patient), amplitude, monopular/bipolar contacts, pulse width, recording 8 lead contacts, and 2-8 EMG channels per patient. Motor evoked potentials are an accurate biomarker for DBS-induced side effects. Meta-active learning can optimize quickly in simulation and the present disclosure can have software system 100 for real-time recording and simulation.

Long-term project outcomes can include shortening DBS programming time by pre-suggesting optimal parameters; having a scalable solution for more complex DBS devices; and programming for other diseases with poor behavioral feedback such as epilepsy, other movement disorders, neuropsychiatric DBS. The present disclosure can finalize model training in silico, deploy in patients, and compare optimal parameters to post-operative programming. A few findings include 1) motor evoked potentials are an accurate biomarker for DBS-induced side effects; 2) meta-learning can accurately model population-level DBS responses; and 3) meta-active learning can quickly find optimal parameters in simulation.

In some embodiments, the modeling population level DBS data with meta-learning for simulating parameter selection can include architecture with a variational inference-based approach. It can maximize lower bound on mutual information between learned embedding and patient response to DBS stimulation and capture heterogeneity amongst patients while simultaneously learning form the entire population. To maximize the mutual information between the embedding and the biomarker's prediction, which is what quantifies the uncertainty in the embedding after seeing the biomarker, the present disclosure can use the MSE (embedding_hat, embedding) in the VI pair's backpropagation. Predictor and embedding estimator networks are fully connected layers with relu activations. In design considerations, there can be DBS parameter encoding strategies including One-hot (original idea) (9-D vector for contacts, where 1=assignment as cathode and −1=assignment as anode; Amplitude and pulse width normalized and appended to input; and 11-D input space) Ordinal/binary (Encode contact depth and segments in 3-D for the cathode, append the same structure for the anode (6-D total); One dimension with ordinal values for the contact number (1-4, case takes 0); Two dimensions with binary values per segment (A=0,0; B=0,1; C=1,0; full ring=1,1); Amplitude and pulse width normalized and appended to input; and 8-D input space) and ordinal (basic) (Encode contact with segments as one dimension (C=0, 1=1, 2=2A, 2B=3, . . . , 4-10) for cathode, append the same structure for the anode (2-D total); Amplitude and pulse width normalized and appended to input; and 4-D input space). Additional design considerations can include embedding size (Investigate 2D through 5D) and biomarker representation (Current: max DLEP value across recorded contacts, averaged z-scored amplitude across recorded muscles; Alternatives for easier learning: average of log-transformed muscle amplitudes, count of responding muscles). Some performance considerations are input dimensionality: smaller D while maintaining accuracy should give better RL performance. Additionally, to investigate correlation to the embedding space with the lead location in the brain, given each patient's embedding and lead position from modeled MRI data. The embedding smoothness i. Seems reasonable; evaluated by generating a line of embedding coordinates and evaluating similarity of parameter grid responses against nearest patients. In embedding sampling, the present disclosure can consider uniform sampling from min/max embedding performs poorly and alternatives: GMM, bounded polygon, using patient coordinates with added noise. In evaluating, the present disclosure can leave-one-out cross-validation loss for different encoding strategies, embedding dimensions.

With training, the present disclosure can consider training (reward shaping and hyperparameters). With reward-shaping: Current reward: maximize improvement in brain model error for selecting given parameter combination; Exploitation: DLEP+c*MEP (c=2 for now); exploration/exploration tradeoff: reward based upon both improvement in model error and improvement in error in best parameter. During evaluation, the present disclosure can consider the metrics and other potential issues. For metrics: in test time simulation for new patients: the number of trials needed to achieve low % error in finding optimal stimulation parameter and the number of trials needed to find top N percentile of best stimulation parameters. Other issues can include, for first reward function (maximize brain model error)—issues overfitting/testing the same set of parameters multiple times in a row.

Deep brain stimulation (DBS) can be an effective FDA-approved treatment for Parkinson's disease, with more than 200,000 implanted patients globally. The DBS devices disclosed herein can be any DBS devices known in the art for stimulating one or more portions of a user's brain. In some embodiments, the DBS devices can include one or more electrodes or other electromagnetic members configured to deliver electromagnetic energy to portions of the user's brain. To achieve effectiveness, the device parameters settings that can control how electric current is delivered to the patient's brain can be selected manually for each patient over a months-long process by a trained clinician, delaying optimal clinical outcomes. The present disclosure is developing a fully automated system 100 that can optimize DBS parameters for Parkinson's disease. This system 100 can consist of multiple key features: (1) The use of neural biomarkers recorded from the DBS device and electromyography can provide faster, more precise evaluation of parameters compared to manual evaluation. (2) An AI simulation platform that can use meta-learning to model biomarker data collected from a population of patients, that can allow for training of AI solutions to automatically select parameters. (3) Meta-active learning (meta-AL), an AI method that can optimally apply stimulation parameters, evaluate based on the resulting neural biomarkers in real-time, and iterate to most quickly optimize parameters in new patients. The combination of these methods can provide a patient-specific list of best stimulation parameters, providing earlier improvement in patients' quality of life by reducing the amount of time that can be needed to select the patient's device settings.

Deep brain stimulation (DBS) parameters can be selected manually for each patient in a months-long trial-and-error process, delaying optimal clinical outcomes. The present disclosure is developing a fully automated approach that can optimize STN-DBS parameters for Parkinson's disease intraoperatively. Our platform can use meta-active learning (meta-AL), an AI method that leverages a database of prior PD patients to learn how to optimally apply stimulation parameters, measure the effect on neural biomarkers in real-time, and iterate to most quickly optimize parameters in new patients. Its optimization goal is to maximize DBS local evoked potentials (DLEP) recorded from the DBS lead while minimizing EMG-measured motor evoked potentials (mEP) electrophysiological biomarkers of pathway activation for symptom relief and side-effects, respectively.

To evaluate the relationship between mEP and side-effects, the present disclosure applied 319 different stimulation settings at both low (<30 Hz) and high (130 Hz) frequency and respectively measured mEP's on multiple facial/limb muscles (2-6 channels per patient) and patient-reported motor side effects for each setting. The present disclosure similarly computed DLEP and mEP biomarkers for low-frequency stimulation data collected from 52 patients during DBS implantation surgery (total 1364 settings).

The present disclosure can show that the presence of mEP can predict which DBS parameters induce motor side-effects (86% accuracy), that the muscles displaying mEP are consistent with side effect location, and the present disclosure can validate an automated mEP detection algorithm (92% accuracy vs. visual detection). The present disclosure then can apply meta-learning to predict DLEP and mEP values as a function of stimulation parameters, showing that a meta-trained neural network can predict biomarker values for unseen parameters from novel patients with low cross-validation error (DLEP: p<0.0001, mEP: p<0.005). The present disclosure can then use this modeled data to provide the basis for a simulation environment where meta-AL can practice DBS programming, showing that meta-AL can learn to efficiently find optimal parameters of simulated patients in silico. Last, the present disclosure can demonstrate an open-source system 100 for real-time closed-loop biomarker recording and parameter selection in Python.

The present disclosure can show that motor evoked potentials are an accurate biomarker for DBS-induced side effects, and that meta-active learning can efficiently find optimal parameters in silico. Our next step is to deploy this method in patients. Long-term, the approach could reduce DBS programming time by providing expected best parameters before clinical testing and could enable effective programming for other DBS applications (epilepsy, neuropsychiatric diseases, etc.) where behavioral feedback is limited.

When a robotic system 100 is faced with uncertainty, the system 100 can take calculated risks to gain information as efficiently as possible while ensuring system 100 safety. The system 100 can safely and efficiently gain information in the face of uncertainty spans domains from healthcare to search and rescue. To efficiently learn when data can be scarce or difficult to label, active learning acquisition functions can intelligently select a data point that, if the label were known, could most improve the estimate of the unknown model. Unfortunately, prior work in active learning suffers from an inability to accurately quantify information-gain, generalize to new domains, and ensure safe operation. To overcome these limitations, the present disclosure can develop Safe MetAL, a probabilistically-safe, active learning algorithm which can meta-learn an acquisition function for selecting sample efficient data points in safety critical domains. Our approach can be a novel integration of meta-active learning and chance-constrained optimization. The present disclosure can (1) meta-learn an acquisition function based on sample history, (2) encode this acquisition function in a chance-constrained optimization framework, and (3) solve for an information-rich set of data points while enforcing probabilistic safety guarantees. The present disclosure presents state-of-the-art results in active learning of the model of a damaged UAV and in learning the optimal parameters for deep brain stimulation. Our approach can achieve a 41% improvement in learning the optimal model and a 20% speedup in computation time compared to active and meta-learning approaches while ensuring safety of the system 100.

The present disclosure can demonstrate the advantage of Safe MetAL across two domains: 1) a high-dimensional damaged UAV domain and 2) a novel DBS domain, both safety critical environments in which sample efficiency is of utmost importance. Our approach can outperform previous Bayesian, meta-learning, and active learning approaches in terms of expected informativeness, safety, and computation time.

The present disclosure presents Safe MetAL, a meta-learning algorithm for learning a domain-specific acquisition function that can accurately quantify expected informativeness. Safe MetAL (1) can meta-learn an acquisition function to quantify domain specific expected informativeness of a data point without hand-derived features and ad hoc engineering, and (2) reasons about exploitation vs. exploration by trading off gaining information and probabilistically-safe control.

The present disclosure can formulate a novel bridge between deep learning and mathematical programming techniques in a way that is fully, end-to-end differentiable and trainable by embedding this meta-learned acquisition function within a chance-constrained optimization framework to achieve probabilistic guarantees. The present disclosure can show that the approach can generalize across two disparate domains and set a new state-of-the-art for increase in model accuracy (41%) compared to Bayesian, active and meta-learning approaches and computational speed (+20%) versus two active and meta-learning baselines while also providing probabilistic advantages.

The present disclosure describes the problem set-up via a motivating example: learning the dynamics model of a damaged UAV. In this example, the objective is to safely and efficiently learn the altered UAV dynamics, \hat{ƒ}_{ψ}, and maintain controllability of the system 100 despite damage. UAVs are susceptible to a range of failure scenarios that are difficult to predict and model, and, when damaged, UAVs can have tight time constraints for recovery. Specifically, the present disclosure can seek to determine the action the UAV can take next to provide maximum information about the nature of the damage given the UAV's previously experienced states and actions without going into unsafe configurations. To do so, the present disclosure can learn a function that describes the expected informativeness of taking any action, conditioned on the prior experience of the UAV and subject to safety considerations. The present disclosure can set up the problem in three steps: 1) active learning, 2) safety, and 3) meta-learning.

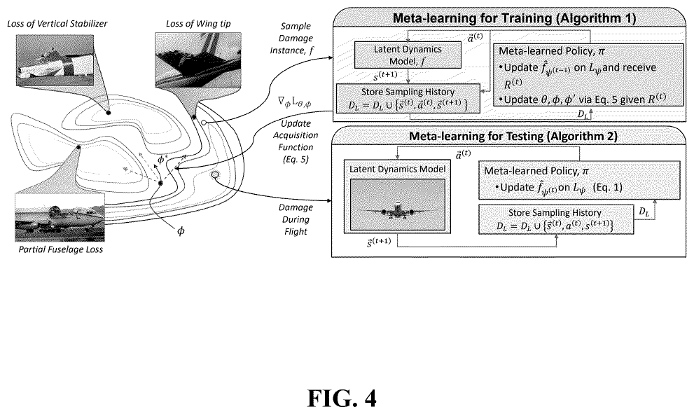

First, the present disclosure can define the unlabeled dataset, D_{U}= [symbol] \vec{s}⁽ⁱ⁾,\vec{a}⁽ⁱ⁾ [symbol] ᵢ₌₁ⁿ, as being able to consist of possible state-actions pairs that the UAV could potentially experience and the labeled dataset, D_{L}= [symbol] \vec{s}⁽ⁱ⁾,\vec{a}⁽ⁱ⁾,\vec{s}⁽ⁱ⁺¹⁾ [symbol] ᵢ₌₁^{m}, as the set of state transition can triple experienced by the UAV in flight. \vec{s}^{(t+1)} can be the state that results from applying action \vec{a}^{(t)} in state \vec{s}^{(t)} at time t as governed by the latent dynamical model, ƒ (FIG. 4).

Our Long Short-Term Memory (LSTM) neural network, with parameters θₜ, can learn an encoding of sample history, z^{(t)}=ε_{θ}(S^{(t)}). This sample history through time, t, can be defined as S^{(t)}=(\vec{s}⁽⁰⁾, \vec{a}⁽⁰⁾, \vec{s}⁽¹⁾, . . . , \vec{a}^{(t-1)}, \vec{s}^{(t)}) which the present disclosure refers to as the meta-state. Our acquisition function, \dot{Q}_{ϕ}: [symbol] ×Z→ [symbol] , can learn to map a candidate action, â, to a measure of expected informativeness conditioned on the embedding of sample history, \vec{z}. This problem can setup corresponds to a Partially Observable Markov Decision Process (POMDP), where the observations are the samples, \vec{s}ᵢ and the state can describe the latent dynamics (i.e., the transition function, ƒ) with actions, \vec{a}, discount factor, γ, and reward function, R, described below. The present disclosure cannot have access to the observation function, Ω. The present disclosure can convert this POMDP to a Markov Decision Process (MDP) in which the present disclosure uses function approximation to (1) learn a compact representation, z^{(t)}, of the history of observations via ε₀ and leverage this representation to (2) train a history-dependent Q-function, Q_{ϕ}.

The present disclosure can utilize expected informativeness (i.e., improvement in model accuracy due to the addition of new observations to the training set) as the reward signal for training the network. To determine the decrease in model error, the present disclosure creates a dataset, D^{Test}, by sampling from the known dynamics model, which the present disclosure can have access to during training. The reward signal, R^{(t)}, which is defined in (2), is the decrease in model error when applying action, \vec{a}^{(t)}, in state, \vec{s}^{(t)}, and experiencing state, \vec{s}^{(t+1)} (i.e., D_{L}∪ [symbol] \vec{s}^{(t)}, \vec{a}^{(t)}, \vec{s}^{(t+1)} [symbol] ). Intuitively, a large reward means that the present disclosure can have selected an action that greatly decreases the error of the dynamics model, \hat{ƒ}_{ψ}. ψ is the parametrization of \hat{ƒ}_{ψ}_{^{(t)}} at time, t.

Second, the present disclosure can incorporate safety when selecting the optimal action. The present disclosure can consider the system 100 to be safe if there is a high probability of the system 100 returning to a safe volume, which the present disclosure can discuss further in Section III. Therefore, the present disclosure can encode the acquisition function into a mixed-integer linear program (MILP) which allows us to impose safety constraints and can choose the set of actions which maximize expected informativeness, while also ensuring safety.

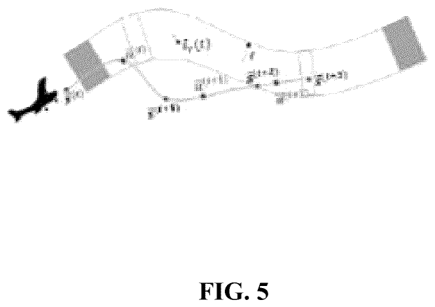

In the formulation, chance-constraints can allow us to model uncertainty and ensure the probability of failure remains under a certain threshold. Thus, by utilizing a chance-constrained MILP, the present disclosure can efficiently arrive at a solution for non-convex optimization problems while also providing probabilistic guarantees. The present disclosure can transform each piece-wise term in the acquisition function into a set of integer, linear constraints via the “big M” method. The present disclosure can solve the chance-constrained MILP via linearization techniques discussed. While limited prior work has explored safety and chance constraints for learning and control, the present disclosure can go beyond this prior work by taking into account the effect that querying a label has on the underlying system's ability to remain in a safe configuration. In the damaged UAV and DBS domains, choosing a sequence of unsafe actions can lead to the UAV crashing or an ictal state in the brain. As depicted in FIG. 5, the present disclosure can assume a set of known safe states (e.g., level flight above the ground for a UAV) and the present disclosure can allow the system 100 to deviate from a safe region temporally to gain information provided that the system 100 has a sufficient probability of returning to a safe state. The present disclosure can elaborate on how these safe states are identified and the external validity. To approximate the uncertainty of the states, the present disclosure can assume the model error comes from a Gaussian distribution with a known mean and variance calculated via the bootstrapping method described.

Finally, the present disclosure can seek to enable the system 100 to generalize beyond a single active learning task (e.g., damage to a specific part of the UAV) to a broader class of tasks (i.e., any type of damage). The present disclosure can aim to learn this acquisition function without hand-engineering features or heuristics. Therefore, the present disclosure can incorporate meta-learning to train the acquisition function, Q_{ϕ}, and embedding of previously experienced states and actions, ε_{θ}. The present disclosure can train Q_{ϕ} over a distribution of optimization problems (e.g., loss of vertical stabilizer, wing damage etc.) to enable Q_{ϕ} to generalize to an unforeseen damage scenario.

### Safe Meta-Learning Architecture

Our architecture can consist of three key components: 1) an LSTM-based representation of sample history, 2) a meta-learned acquisition function that accurately quantifies expected informativeness, and 3) safety constraints imposed via the linear program. An overview of the architecture is shown in FIG. 4 and is described below.

### Policy

Our policy (Eq. 3 below) can be determined by maximizing both the probability of the system 100 remaining in a safe configuration and expected informativeness along the finite trajectory horizon, [t, t+T). Therefore, the policy can select the set of actions, \vec{a}^{(t:t+T)}, which maximizes both safety and expected informativeness. The present disclosure can linearize the objective function following the linearization procedures.

$$\begin{matrix}{a}^{( t + T )} = π ( {ℰ}_{θ} ( {𝒮}^{( t )} ) ) \\ = {arg max}_{{\vec{a} ( \text{?} + x )}_{ϕ}} λ [ {Q}_{ϕ} ( {\vec{a}}^{( \text{?} + T )} , {\vec{z}}^{( t )} ) ] + ( 1 - λ ) [ 1 - ϵ ]\end{matrix} \qquad ( 3 )$$

$$\text{?} \text{indicates text missing or illegible when filed}$$

subject to

$$1 - ϵ \leq Pr \{ {‖ {\vec{s}}^{( t + T )} - {\vec{s}}_{r} ( t ) ‖}_{1} \leq \vec{r} \} \qquad ( 4 )$$

Q_{ϕ}(\vec{a}^{(t:t+T)}, \vec{z}^{(t)}) describes the expected informativeness along the trajectory when the set of actions, \vec{a}^{(t:t+T)}, is taken in the context of the sample history encoding, z^{(\vec{t})}, Π is the chance-constrained policy which selects an action that the UAV should take to maximize both expected informativeness and safety. The LSTM neural network, ε_{θ}, can map the sample history, [symbol] ^{(t)}= [symbol] \vec{s}^{(0:t)}, \vec{a}^{(0:t)} [symbol] , (i.e., previously experienced states and actions), to the encoding \vec{z}^{(t)}. λ can be a hyper-parameter that allows us to adjust the trade-off between safety and expected informativeness while still guaranteeing a minimum level of safety. Properly balancing λ, as in any multi-criteria optimization problem, requires domain expertise. The estimated probability of remaining in a safe configuration can be 1−ϵ, where ϵ∈[0, ϵₘₐₓ] and 1−ϵₘₐₓ can be the minimum acceptable safety level. The present disclosure provides more details on safety in the following section.

### Definition of Safety

Next, the present disclosure details the safety constraints which are enforced via the MILP. The present disclosure can define a volume of safety, as depicted in FIG. 5, around a desired reference trajectory, \vec{s}ᵣ(t), and enforce the constraint that the system 100 must be able to return with probability 1−ϵ to a state, \vec{s}_{t+T}, at time t+T, such that \vec{s}_{t+T} is within this volume of safety. Intuitively, this means that the UAV will be able to take an action outside of the volume of safety to gain information but must return to a safe state at time t+T. Mathematically, the present disclosure can define this safety constraint. \vec{s}ᵣ can define a safe state (e.g., straight and level flight for a UAV), and \vec{r} can be the radius encompassing all known safe system states. The radius of the volume can be a hyperparameter defined by the user and requires domain expertise to determine. The closer the user wants the system 100 to remain to the nominal safe state, the smaller the radius can be. 1−ϵ can be the probability of remaining in the safe region. This volume can be converted into linear constraints, thus creating a convex optimization problem. Leveraging such a pre-defined safety envelope can be consistent with prior work in safe robotics and chance-constrained optimization. By formulating the safety constraints in this way, the present disclosure can guarantee a minimum probability of safety while simultaneously optimizing for additional safety and expected informativeness. In other words, the MILP can select an action that meets the minimum safety requirements, and if possible, can select an even safer action than the minimum specified level of safety, all other things being equal.

### Meta-Learning

To infer the acquisition function, the present disclosure can meta-learn over a distribution of related tasks, which, in the motivating example, consist of various damage modes of the UAV (e.g., wing damage, actuator damage, etc.) as shown in FIG. 4. By meta-learning over this distribution, the present disclosure can construct an acquisition function that accurately defines the expected informativeness of an action when learning the unknown UAV dynamics model.

The acquisition function, Q_{ϕ}, can be trained via Deep Q-Learning with target network, Q_{ϕ′}, which has been shown in prior work to improve training stability. The learned acquisition function, Q_{ϕ}, is utilized by the MILP policy, which selects the optimal actions, \vec{a}^{(t:T)}, subject to safety-constraints. The reward, R^{(t)}, for taking a set of actions in a given state is defined as the decrease in the MSE error of the model, ƒ_{ψ}_{^{(t)}}, achieved by adding training data, [symbol] \vec{s}^{(t)}, \vec{a}^{(t)}\vec{s}^{(t+)} [symbol] , to D_{L}. The Q-function is trained on a set of optimization problems drawn from a distribution of similar black-box functions to minimize the Bellman Residual.

$${ℒ}_{θ , ϕ} = {( {R}^{( t )} + \text{?} {Q}_{ϕ} ( π ( {ℰ}_{θ} ( {𝒮}^{( t + 1 )} ) ) , {\vec{z}}^{( t + 1 )} ) - {Q}_{ϕ} ( {\vec{a}}^{( t )} , {\vec{z}}^{( t )} ) )}^{2}$$

$$\text{?} \text{indicates text missing or illegible when filed}$$

This Bellman loss of the Q-function is backpropagated through the Q-function in the MILP and through the LSTM encoder, ε_{θ}. The dynamics model, \vec{ƒ}_{ψ}_{^{(t)}}, is retrained with each new set of state-action pairs.

### Algorithm

Algorithm 1 describes the training procedure. For each episode, the present disclosure can sample from the distribution of altered dynamics and limit each episode to the number of time steps, M, tuned to collect enough data to accurately learn the dynamics. At each iteration, the present disclosure can select \vec{a}^{(t)} via the MILP objective and execute the action to observe the resultant state, \vec{s}^{(t+1)}. Our dynamics model, \vec{ƒ}_{ψ}_{^{(s)}}, can be retrained by minimizing the MSE. After observing the reward, the present disclosure can update the Q-function via a sampled batch of transitions.

Algorithm 2 describes how the present disclosure can perform the online, safe, active learning. Intuitively, the algorithm initializes a new dynamics model to represent the unknown or altered dynamics, and the present disclosure can iteratively sample information rich, safe actions via the MILP policy, update \vec{ƒ}_{ψ}_{^{(t)}}, and repeat. The present disclosure can assume at test time that the unknown model comes from the same distribution as the training models.

### Experimental Evaluation

The present disclosure compares Safe MetAL against several baseline approaches in two experimental domains described below.

### High-Dimensional UAV Domain

Safe control of damaged UAVs is a difficult problem in robotics due to the tight time constraints and nonlinear dynamics. The present disclosure can test the algorithm's ability to learn the non-linear dynamics of a UAV before the UAV enters an unrecoverable configuration (e.g., crashing). Because active learning algorithms can be ineffective in high-dimensional domains, the aviation domain also serves to stress test the algorithm's ability to quickly learn a high-dimensional dynamics model given tight time constraints. The present disclosure can base the simulation on theoretical damage models from prior work describing the full equations of motion, within the Flightgear virtual environment. The objective of this domain is to learn the altered dynamics that results from the damage and to maintain safe flight. The UAV can take an information rich action potentially resulting in a deviation outside of the d-dimensional volume of safety, guaranteeing that the UAV returns to a safe state with probability 1−ϵ via action \vec{a}^{(t+1)} at the end of the planning horizon.

### Deep Brain Stimulation (DBS)

DBS is a cutting-edge approach for treating seizure conditions that cannot be controlled via pharmacological methods. Currently, surgeons employ trial-and-error to find control settings that reduce seizures. However, it can be difficult to find a clear mapping from parameter values to reduction in seizures that applies to all patients, as the optimal parameter settings can depend on placement of the device, the individual anatomy, and other confounding factors. Further, a latent subset of parameters can cause negative side-effects. The present disclosure can create simulation environments based on data from six rats where, at each DBS parameter setting, the cognitive function of a rat can be measured by a “memory score.” Data from each rat can then be dissimulated into many digital twins of the rat, creating a population pool over which the present disclosure can meta-learn. To create these digital twins, the present disclosure can employ a validated in silico procedure in which the present disclosure can bootstrap Gaussian Process models trained on in vivo data of DBS in rats to create a virtual experimental domain. The task is to determine the DBS parameters (i.e., signal amplitude) in the simulation environments that maximize each rat's memory score (i.e., rat's ability to recall the location of objects) without causing unwanted side effects (e.g., memory deficits or seizures) which occur when the memory score drops below zero. The reward signal utilized by the meta-learner can be the percent decrease in error between the predicted and actual optimal parameters. This domain and the established in silico evaluation procedure are described further herein.

### Results

### Baseline Comparisons

To demonstrate that meta-learning can be an important component of the framework and produces results superior to prior work, the present disclosure can benchmark against active learning functions, Epistemic Uncertainty and Maximizing Diversity. These active learning functions are linearized and embedded in the safety constrained framework therefore providing a head-to-head comparison between the meta-learned acquisition function and these active learning heuristics. The present disclosure additionally can benchmark against several Bayesian and meta-learning approaches. The present disclosure empirically can validate that Safe MetAL outperforms baselines in the DBS and UAV domains in terms of its ability to safely and actively learn latent parameters.

Epistemic Uncertainty: Can select the action which maximizes the uncertainty of the model, while also imposing safety constraints via a chance-constrained linear program. Maximizing Diversity [29]—Can select actions which maximize the difference between previous states and actions, subject to safety constraints via a chance-constrained linear program. Bayesian Optimization (BaO): Developed in previous work for the DBS domain (Section IV) and is based upon a Gaussian Process model which attempts to efficiently determine the optimal parameters. Meta Bayesian Optimization (Meta BO): Meta-learns a Gaussian process prior offline. Learning Active Learning (LAL): Meta-learns an acquisition function leveraging hand-engineered features.

### Active Learning

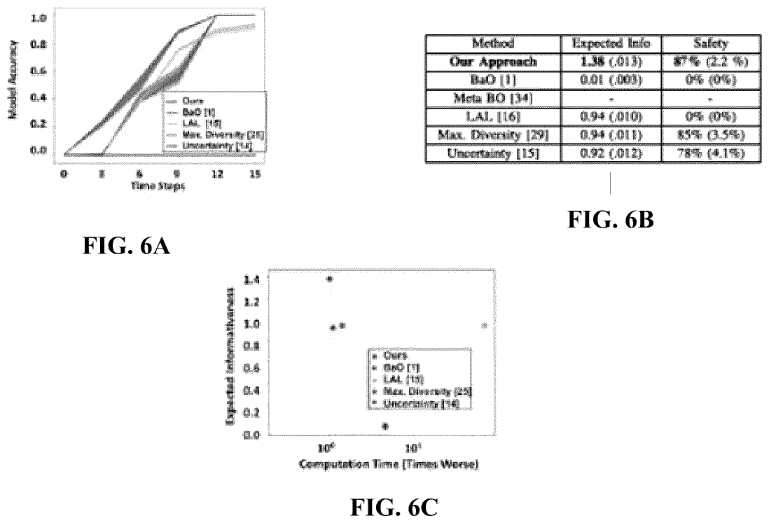

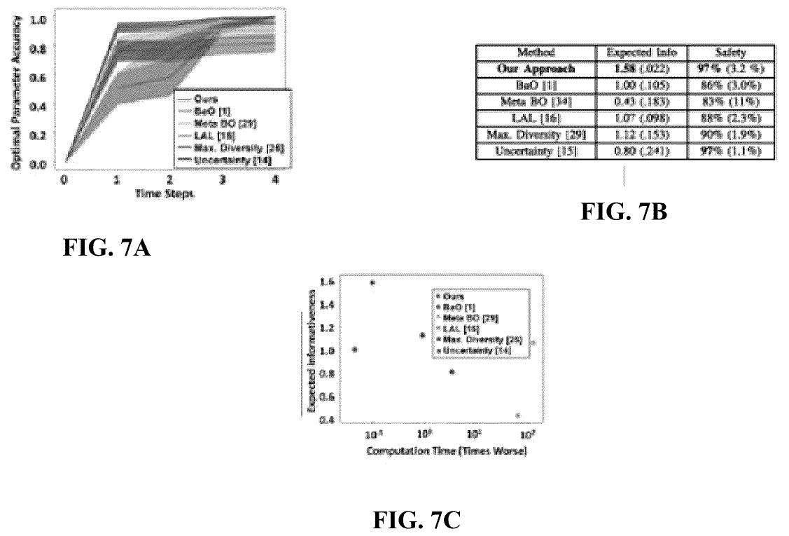

Results from both the UAV and the DBS domains empirically can validate that the algorithm more efficiently learns the optimal parameters (FIG. 7A-C) and system dynamics (FIG. 6A-C) in both domains compared to baseline approaches. The Bayesian baseline, BaO can struggle to learn in the UAV domain, and the present disclosure can find that Meta BO is computationally intractable due to the complexity of the task. Again, Safe MetAL can outperform both active learning heuristics, achieving a 46% improvement over Maximizing Diversity and a 49% improvement over Uncertainty. Safe MetAL can achieve a 47% higher expected informativeness versus LAL.

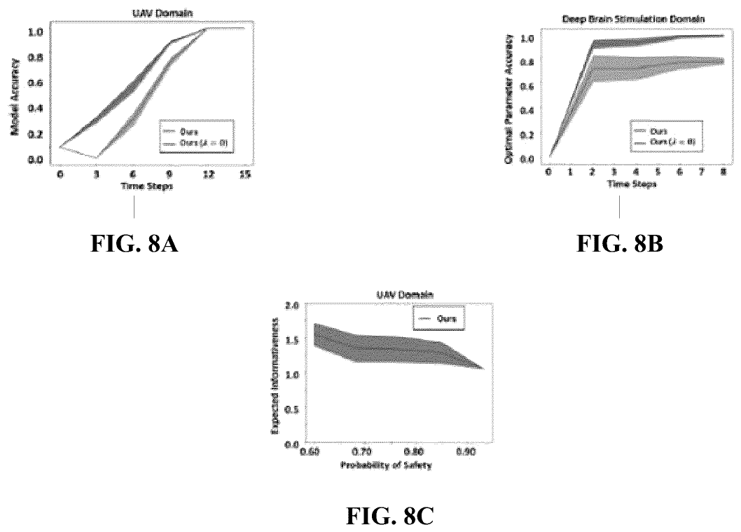

The present disclosure can find similarly positive results in the DBS domain. In this domain, Safe MetAL can select an action that results in 58% higher expected informativeness and a 267% higher expected informativeness on average compared to the two Bayesian baselines, BaO and Meta BO respectively. Compared to the active learning baselines, Maximizing Diversity and Uncertainty, Safe MetAL can perform 41% and 98% better in terms of average expected informativeness respectively. This large increase in expected informativeness that Safe MetAL is able to achieve compared to hand-engineered heuristics, suggests that the meta-learning aspect of Safe MetAL can be important for synthesizing a precise, task-specific acquisition function. Lastly, the present disclosure can show that Safe MetAL can outperform by 47% the meta-learning baseline, LAL, which meta-learns over hand-engineered features. These results demonstrate that the meta-learned embedding is more capable of extracting salient information than the hand-engineered features in LAL. To further verify that the meta-learning aspect of Safe MetAL is necessary for achieving high expected informativeness, the present disclosure can perform an ablation study as shown in FIGS. 8A and 8B.

### Safety

Because Safe MetAL can more quickly learn the optimal parameter settings, it is also able to ensure safe operation to a greater degree than the baselines in both domains. To empirically validate the safety of each algorithm, the present disclosure can perform a Monte Carlo simulation and determine the percentage of the time that the UAV is able to return to the safe region. The present disclosure can find that Safe MetAL can achieve an 87% probability the UAV will return to the safe region (FIG. 6A). As shown in FIG. 6B, Safe MetAL can outperform the baselines simultaneously in safety and expected informativeness in the high-dimensional UAV domain.

In the DBS domain, Safe MetAL can achieve a 6.3% higher guarantee of safety compared to Maximizing Diversity in FIG. 7A-C Safe MetAL can achieve a 98% greater expected in-formativeness compared to Uncertainty and achieves an equivalent safety guarantee. In FIG. 8C, the present disclosure can show the trade-off between the probability of safety as determined by the MILP and the expected informativeness of an action as a result of adjusting λ. This flexibility can allow for greater emphasis on safety in more safety critical domains, whereas in less safety critical domains, these constraints can be relaxed in favor of higher expected informativeness.

### Computation Time

The computation time of active learning algorithms can be of critical importance especially in highly unstable systems such as a damaged UAV. Across both domains, Safe MetAL not only can achieve a more efficient reduction in model error and improvement in expected informativeness, but the present disclosure can also be faster than all baselines in the high-dimensional UAV domain (FIG. 6C). The present disclosure can demonstrate that the present disclosure can be 20% and 29% faster than the baseline acquisition functions, Uncertainty and Maximizing Diversity respectively. The present disclosure also can demonstrate that the present disclosure can be more than 69 times faster than the meta-learning baseline and 78% faster than the Bayesian baseline, BaO.

In the DBS environment (FIG. 7C), BaO has a slight advantage in computation time, but Safe MetAL can trade the time for 58% greater expected informativeness. Additionally, Safe MetAL can be 68× faster than LAL and 61× faster than Meta BO, the two meta-learning benchmarks.

### Related Work

### Active Learning

Active learning acquisition functions can provide heuristics to select the candidate unlabeled training data sample that, if the label were known, would provide the most information to the model being learned. The sample can be selected that the learner is least certain about. The authors can utilize Expected Improvement (EI) heuristic to balance exploration versus exploitation to determine the optimal stimulation parameters in DBS. Prior literature has also investigated on-the-fly active learning and meta-active learning describes the algorithm Learning Active Learning (LAL). The authors can present a meta-learning method for learning an acquisition function in which a regressor is trained to predict the reduction in model error of candidate samples via hand engineered features. Volpp et al. alternatively can consider a Gaussian Process based method to meta-train an acquisition function on a distribution of tasks. Work by Geifman et al. actively learns the neural network architecture that is most appropriate for a given task, e.g., active learning. Pang et al. additionally proposed a method to learn an acquisition function that generalizes to a variety of classification tasks. Yet, this work has been demonstrated for classification.

### Meta-Learning for Dynamics

Prior work has attempted to address the problem of learning altered dynamics via meta-learning. Belkhale et al. investigated a meta-learning approach to learn the altered dynamics of a UAV carrying a payload; the authors train a neural network on prior data to predict environmental and task factors to inform how to adapt to new payloads. Finn et al. presented a meta-learning approach to quickly learning a control policy. In this approach, a distribution over prior model parameters that are most conducive to learning the new dynamics can be meta-learned offline. While this approach provides fast policies for learning new dynamics, it does not explicitly reason about sample efficiency or safety.

### Safe Learning

Prior work has investigated safe learning in the context of Bayesian optimization and safe reinforcement learning. For example, Sui et al. developed the SafeOpt which balances exploration and exploitation to learn an unknown function; however, this approach can make significant limiting assumptions about the underlying nature of the task. Turchetta et al. safely explore an MDP by defining an unknown safety constraint updated during exploration, and Zimmer et al. utilize a Gaussian process for safely learning time series data. Additionally, Nakka et al. introduced Info-SNOC which utilizes chance-constraints to safely learn unknown dynamics. However, these approaches can fail to incorporate knowledge from prior data to increase sample efficiency, limiting their ability to choose the optimal action. Schrum and Gombolay attempt to overcome this problem by employing a novel acquisition function, Maximizing Diversity, to quickly learn altered dynamics in a chance constrained framework. Yet, the hand engineered acquisition function limits the capabilities of this approach.

### Discussion

The present disclosure presents a novel architecture, SafeMetAL, which, unlike previous hand-engineered approaches, can leverage sample history to meta-learn a domain-specific acquisition function for safe and efficient control of an unknown system. Through the empirical investigation, the present disclosure can demonstrate that the meta-learned acquisition function operating within a chance-constrained optimization framework outperforms prior work in active learning, meta-learning, and Bayesian optimization.

Our approach can simultaneously increase expected informative-ness while decreasing computation time. Safe MetAL can achieve a 41% increase in expected informativeness while decreasing computation time by 20% versus active learning and Bayesian baselines in the DBS domain and is more than 60× faster versus meta-learning baselines in the UAV domain. The present disclosure can find that MetaBO is ill-suited for the UAV domain due to its high dimensionality.

Furthermore, the chance-constrained framework combined with higher sample efficiency results in greater probability of safe operation compared to prior work. The safety results for both LAL and BAO in the UAV domain are very poor due to the fact that both lack built-in safety constraints. Taking a single action in the unstable UAV domain that does not comply with any safety guarantees can result in the UAV moving out of the safe region and into an unrecoverable configuration.

The present disclosure additionally can demonstrate state-of-the-art performance in a healthcare domain, demonstrating that the approach generalizes across diverse systems. The present disclosure can be able to outperform all active learning and meta-learning baselines in expected informativeness and safety. The present disclosure thus can demonstrate Safe MetAL's ability to learn the dynamics of a high-dimensional and safety critical UAV as well as the optimal parameter setting for control of a biological system (i.e., the brain) via DBS.

To the best of the knowledge, Safe MetAL is the first architecture to meta-learn an acquisition function for active learning embedded within a chance-constrained program for probabilistically safe control. Further the approach can set a new state of the art over prior work for active learning across two, disparate domains. Our novel, deep learning architecture, can offer a unique ability to learn an LSTM-based embedding of sample history while utilizing the power of deep Q-learning to learn a task-specific acquisition function. Safe MetAL's can be able to optimize both for safety and expected informativeness by embedding the learned acquisition function in a chance constrained optimization framework. With this novel formulation, the present disclosure can demonstrate that Safe MetAL maintains a high probability of safety while also maximizing the expected informativeness based on a learned representation of sample history.

### Limitations and Future Work

Safe MetAL can assume that the safety region is defined by an unchanging volume of safety and that uncertainty over the states is Gaussian. Additionally, Safe MetAL can require data to meta-learn an acquisition function. However, the results demonstrate that Safe MetAL can enable greater expected informativeness and safety when sufficient training data is available. The distribution of scenarios from which the present disclosure can meta-learn over can be determined either by a domain expert or autonomously by a fleet of robots. First, a domain expert could posit various failure modes (e.g., partial wing damage, actuator failure, etc.) and distributions of cases describing possible dynamics for those modes (e.g., dynamics for partial wing loss of 25%, 50%, etc.). These finite set of cases could be artificially expanded through data augmentation, e.g., adding noise to each mode, similar to domain randomization in Sim2Real transfer [18]. Alternatively, a fleet of robots could collect and train on data on all novel situations experienced by any robot. Finally, the present disclosure can hypothesize that Safe MetAL's performance depends on the representativeness of the training data, which will be explored further in future work.

### Exemplary Method

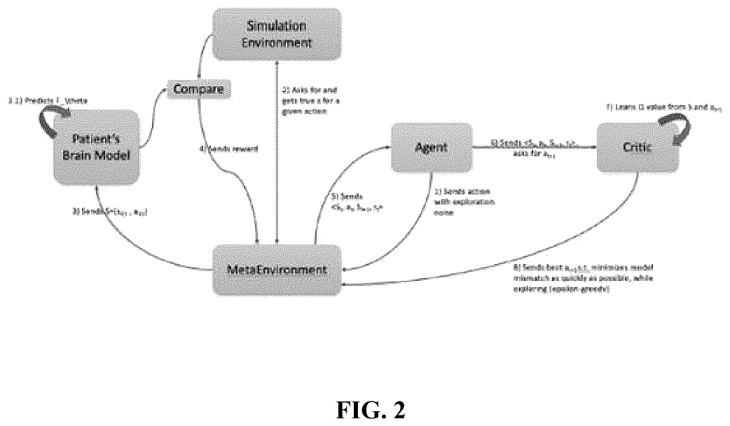

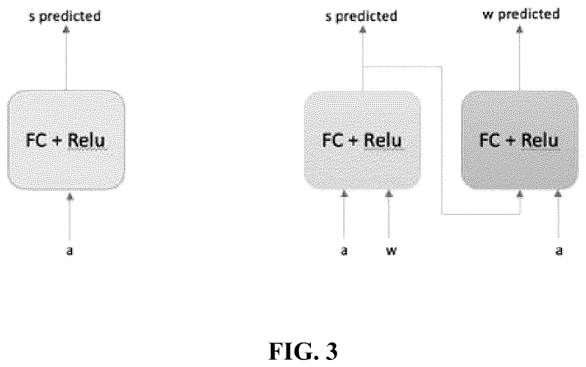

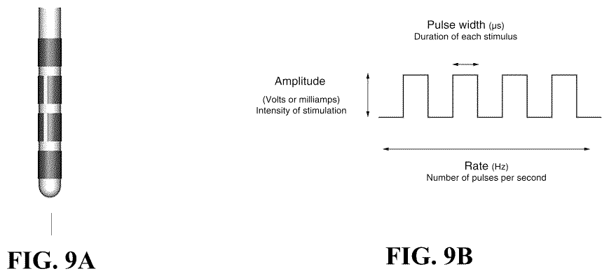

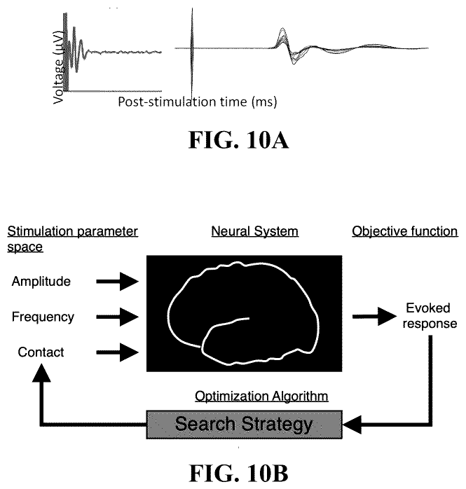

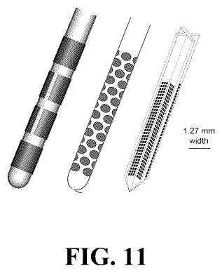

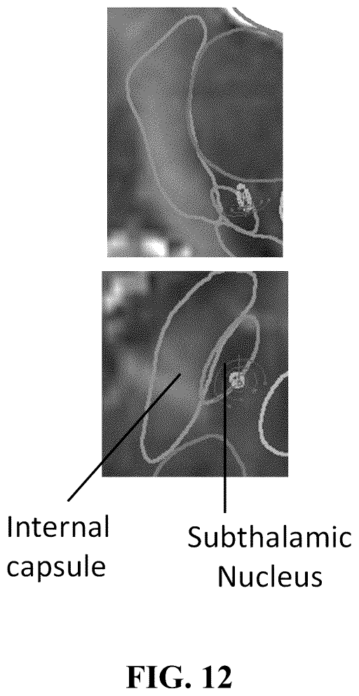

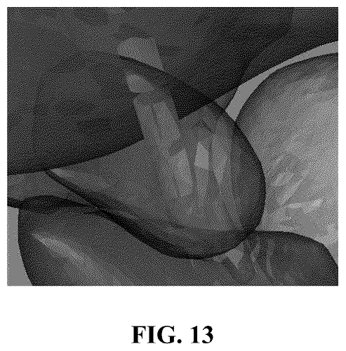

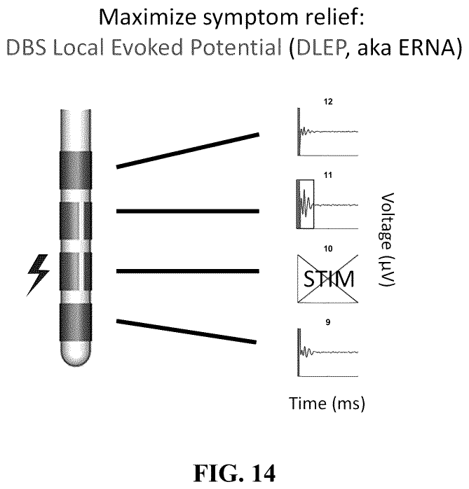

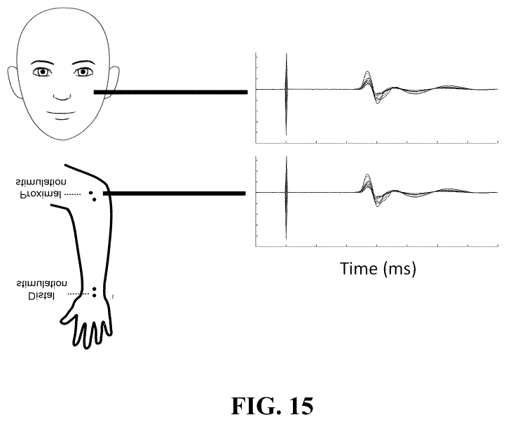

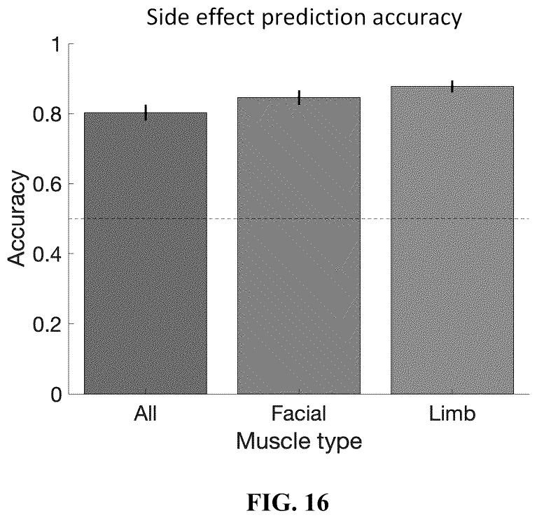

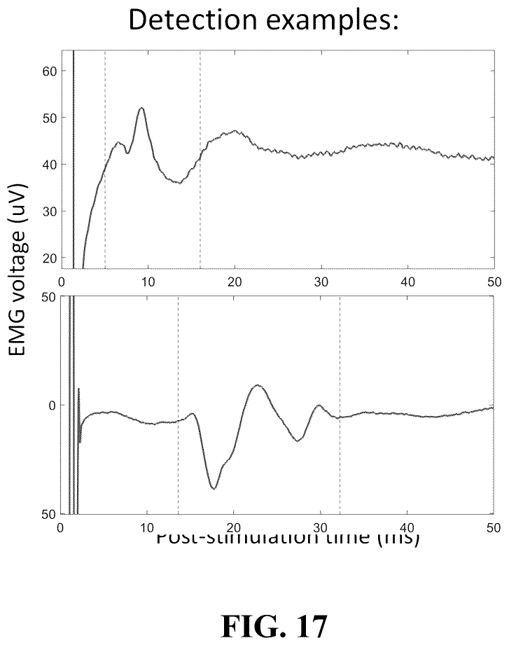

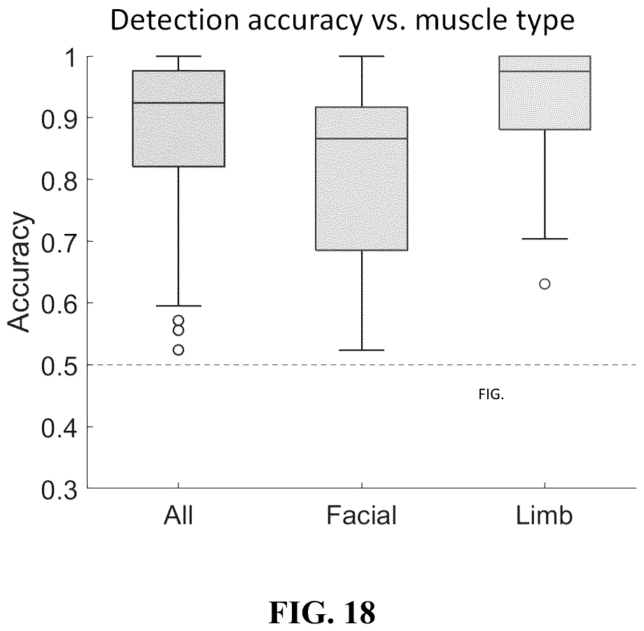

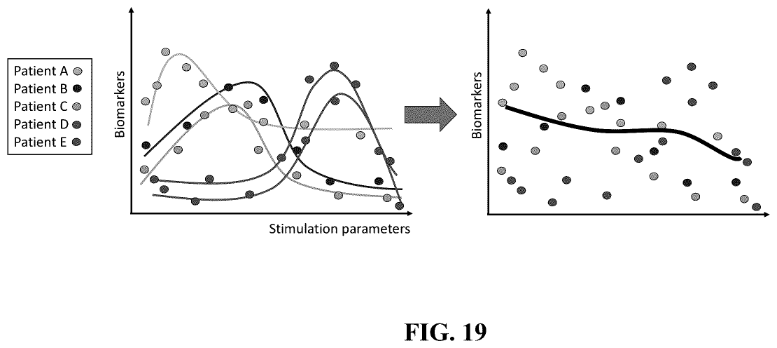

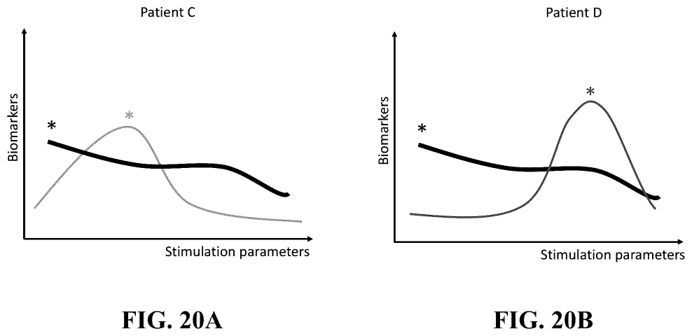

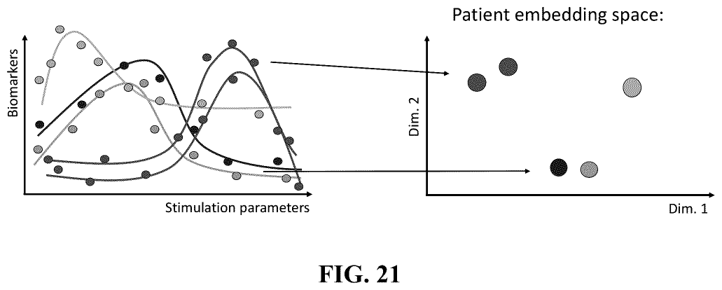

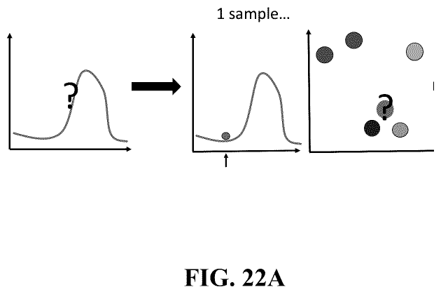

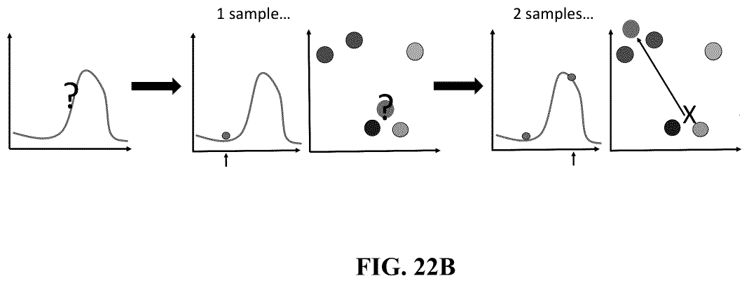

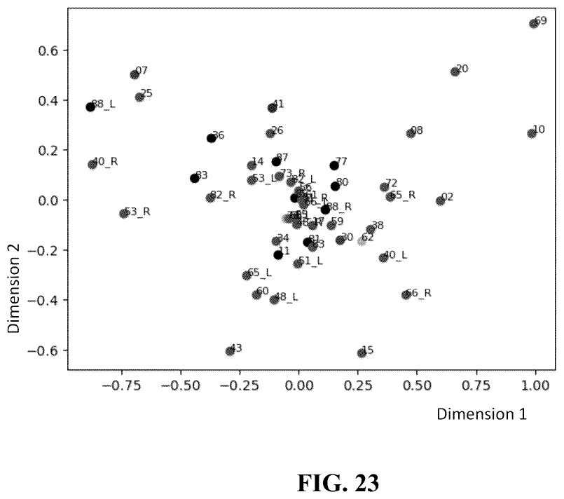

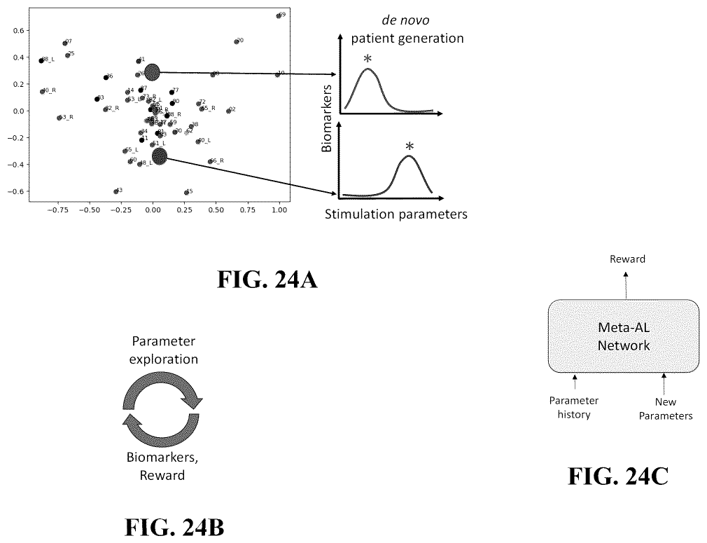

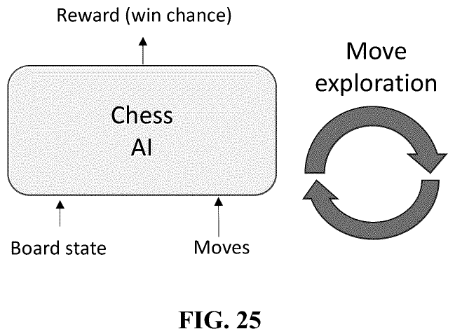

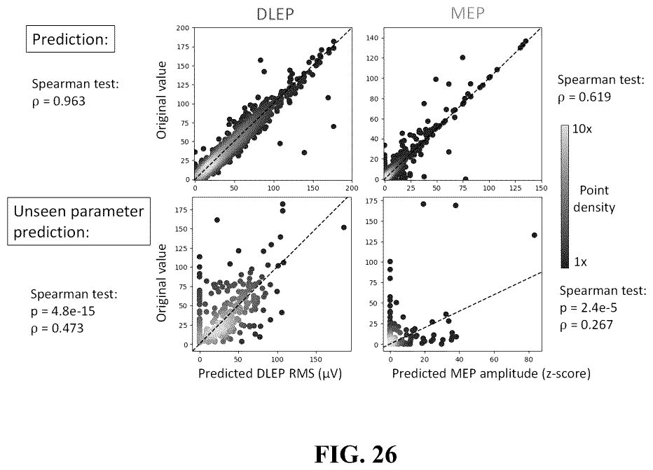

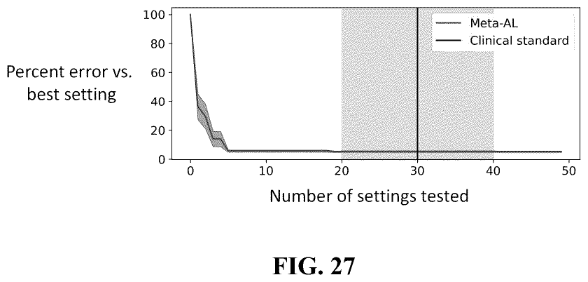

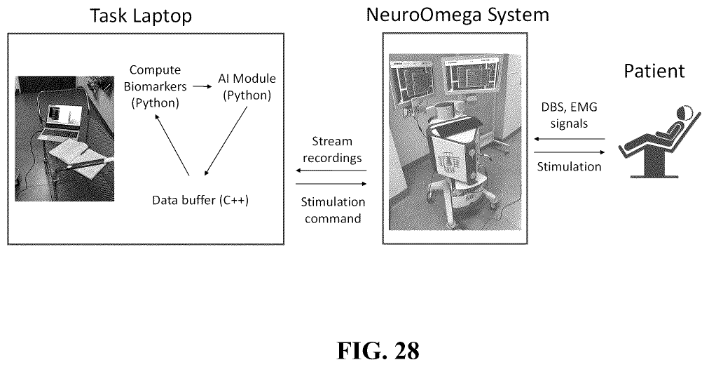

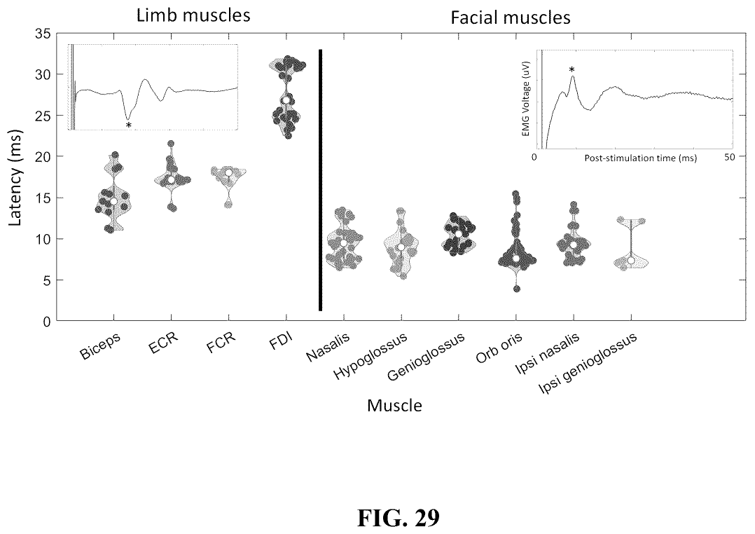

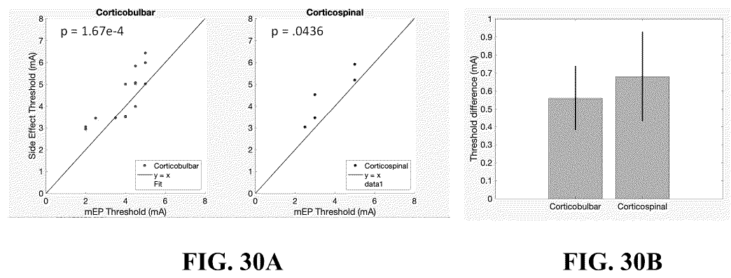

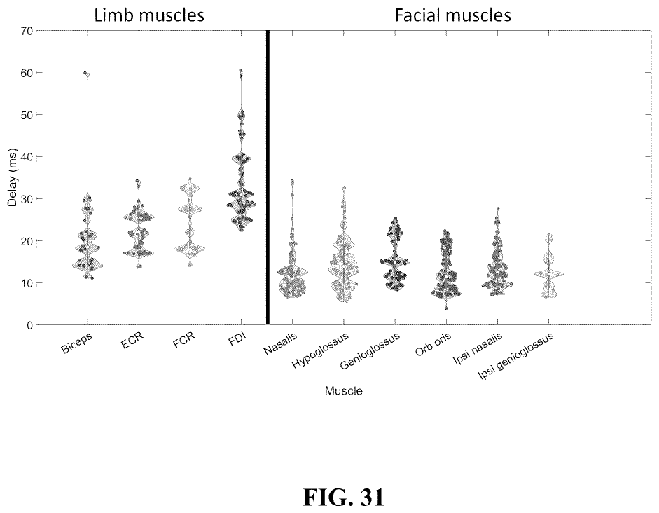

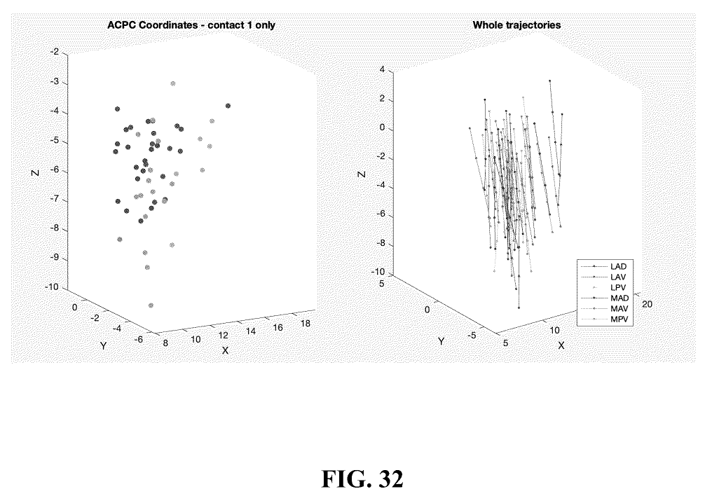

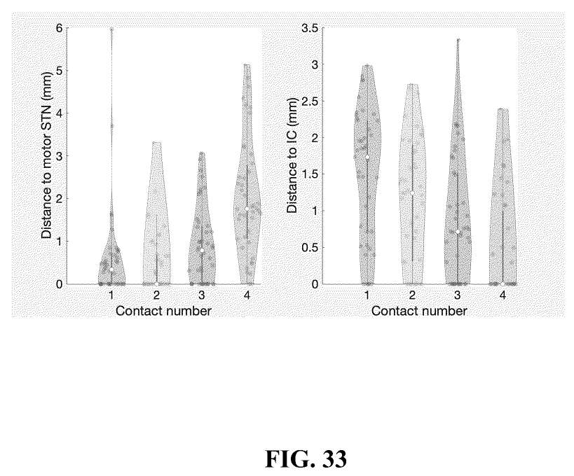

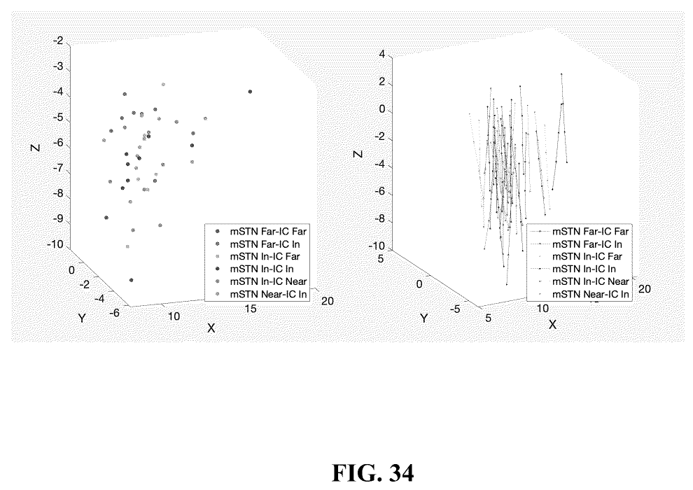

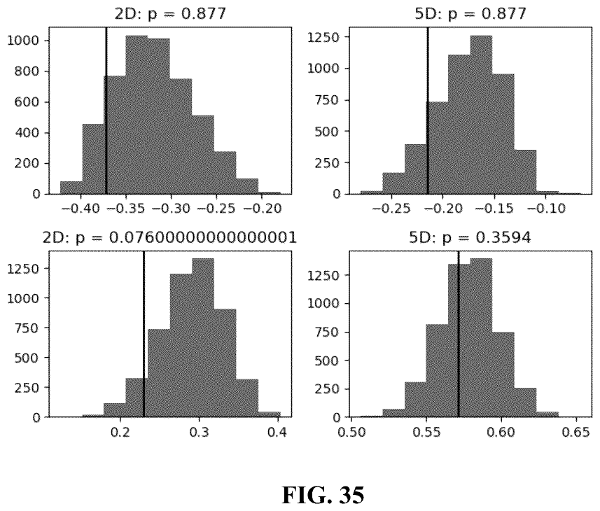

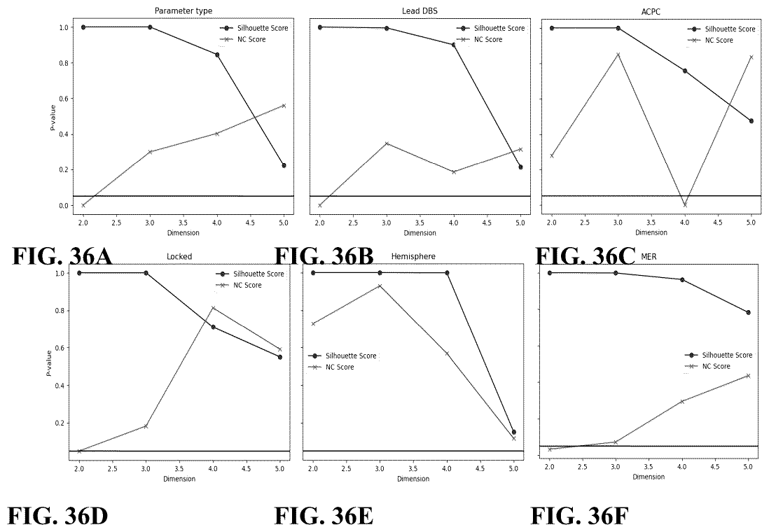

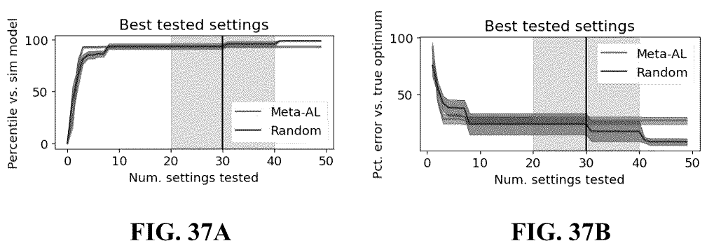

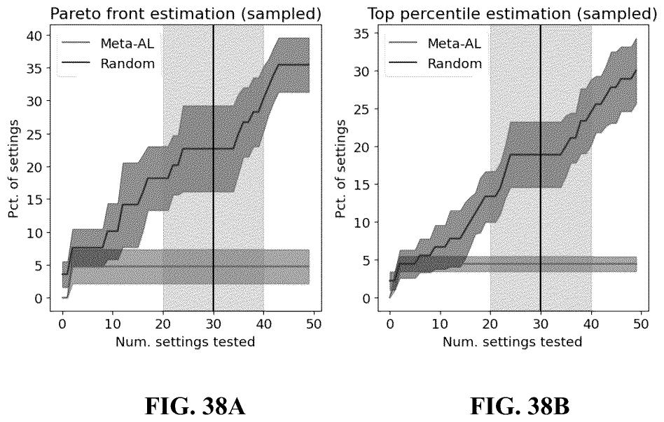

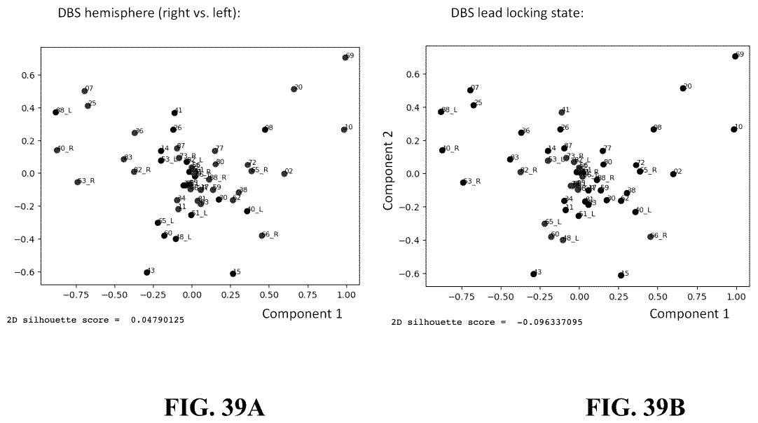

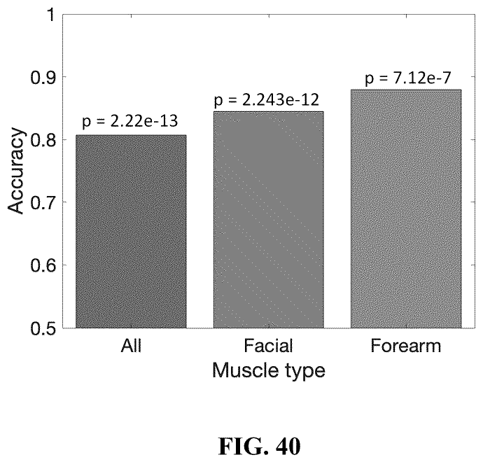

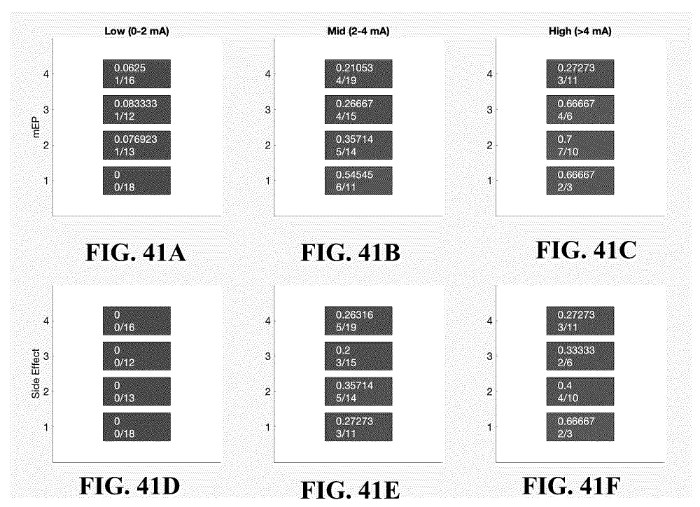

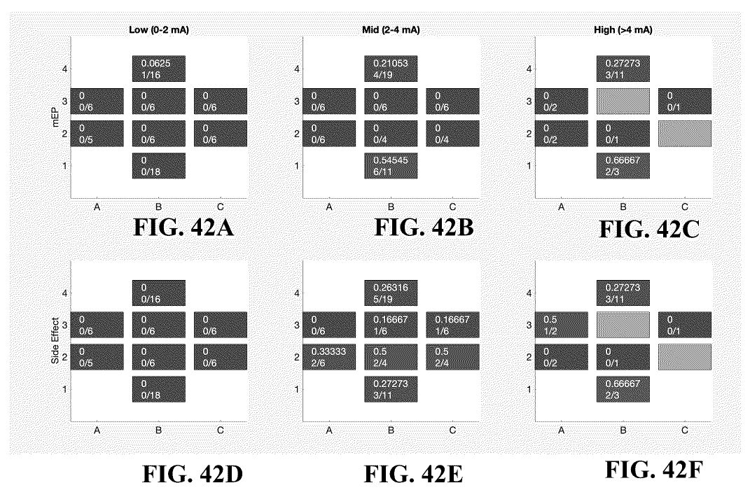

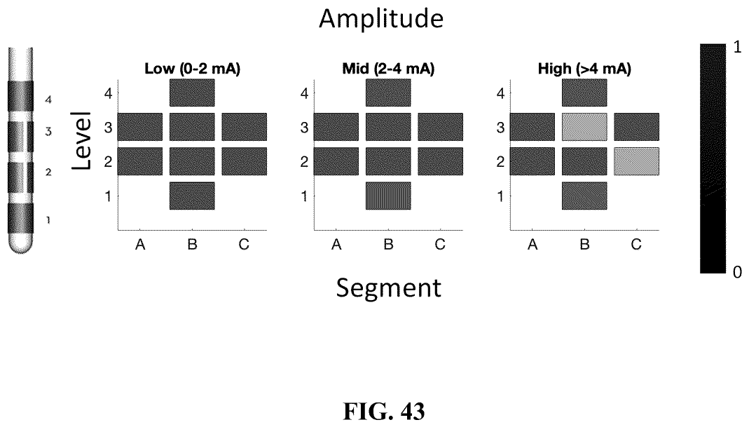

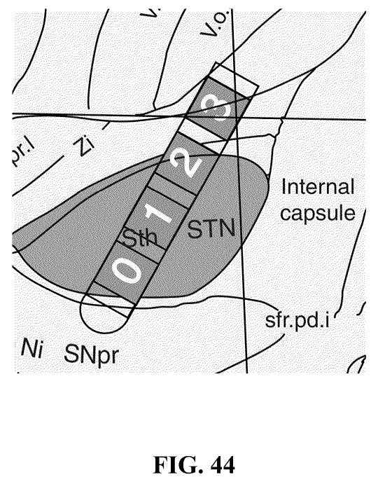

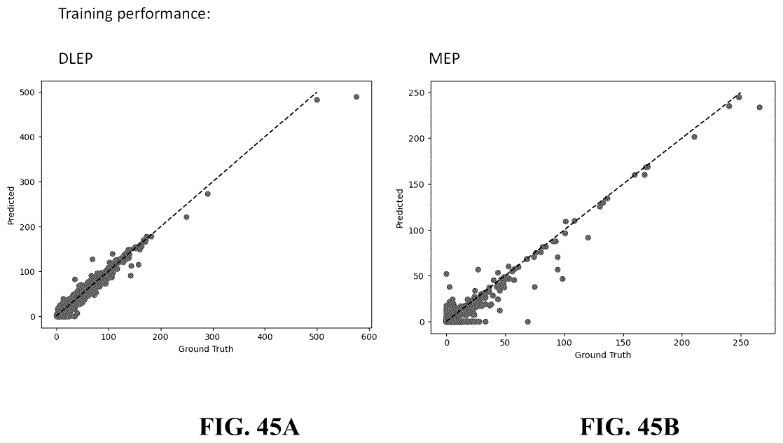

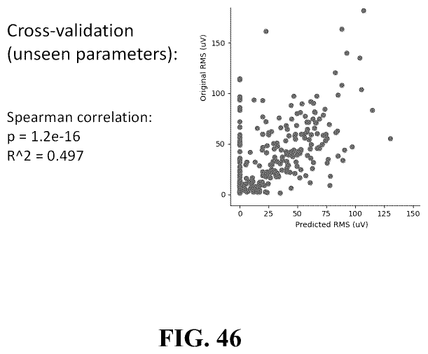

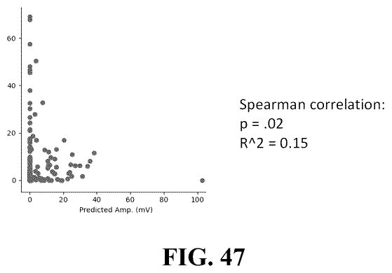

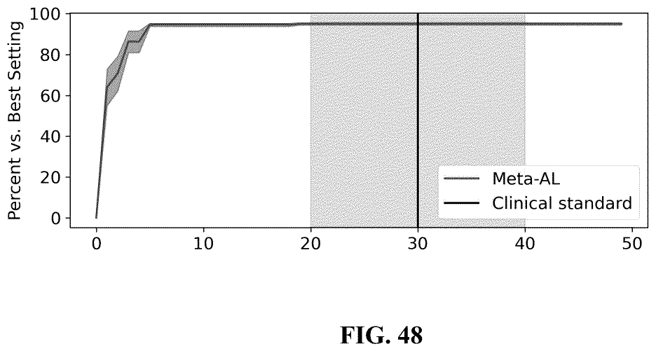

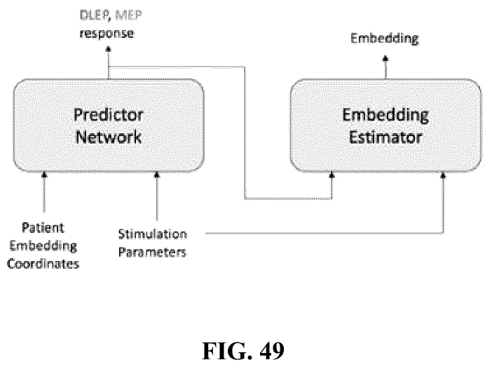

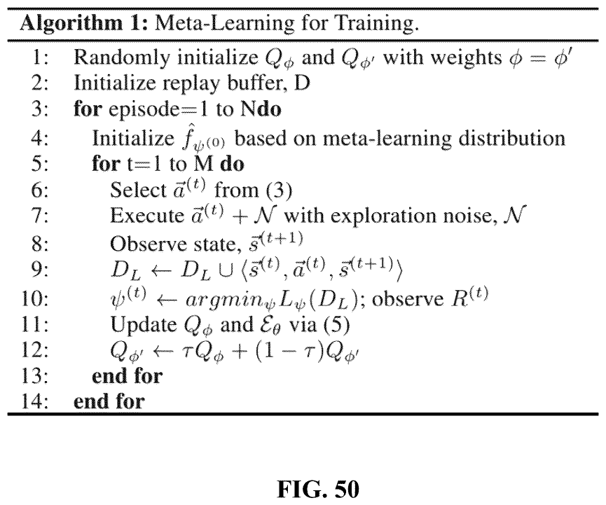

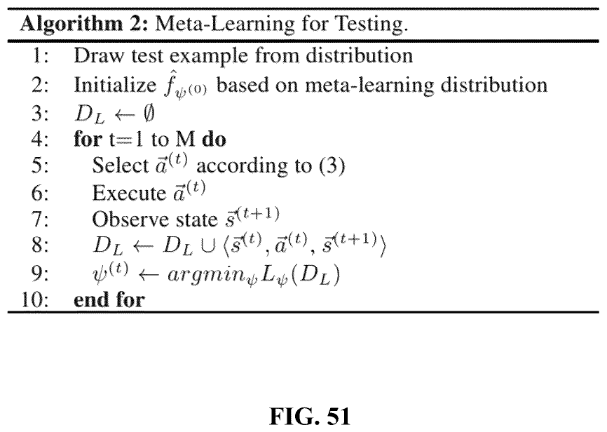

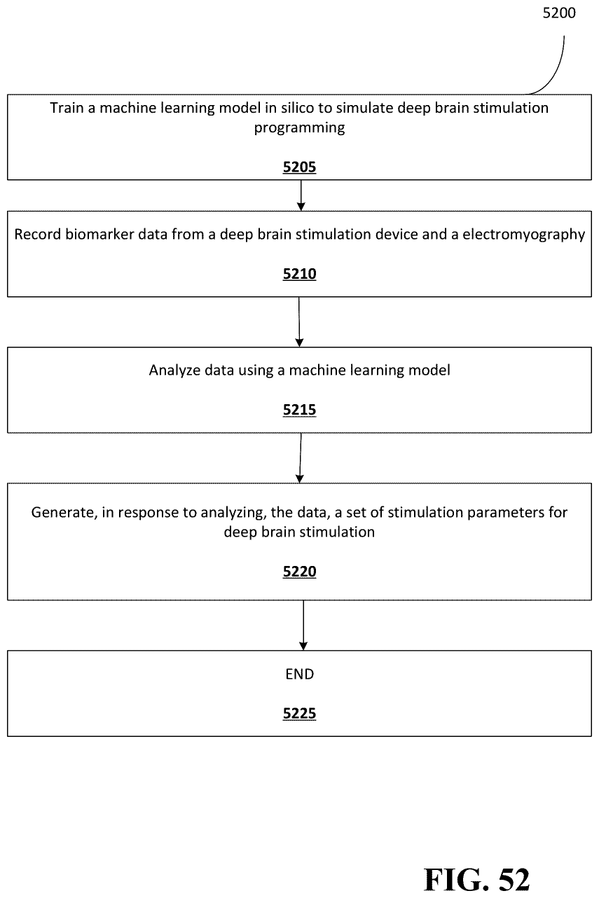

FIG. 52 depicts a flow diagram of an exemplary method 5200 for selecting parameters for a deep brain stimulation device in accordance with various embodiments of the present disclosure. The steps of method 5200 may be performed by one or more components of the system 100, computing environment, and/or controller, as described in more detail with respect to FIGS. 1-52 (and as described above). In step 5205 of method 5200, the system 100 can train a machine learning model in silico (or in a simulation environment) to simulate deep brain stimulation programming. This involves using a dataset of prior patient responses to various stimulation settings to train the machine learning model. The goal is to enable the system 100 to predict optimal stimulation parameters without the need for extensive manual tuning. By simulating DBS programming in silico, the system 100 can efficiently explore a wide range of parameters and identify those that are most likely to be effective for individual patients. This approach, as described above, not only accelerates the programming process but also enhances the precision of parameter selection, potentially leading to better clinical outcomes.

In step 5210 of method 5200, the system 100 can record biomarker data from a deep brain stimulation device (DBS) and an electromyography. The DBS devices disclosed herein can be any DBS devices known in the art for stimulating one or more portions of a user's brain. In some embodiments, the DBS devices can include one or more electrodes or other electromagnetic members configured to deliver electromagnetic energy to portions of the user's brain. During this step, the system 100 can collect critical biomarker data that are essential for optimizing DBS parameters. This includes data recorded from the DBS device itself as well as from electromyography (EMG) sensors (as described above and not repeated herein for brevity). The biomarkers, such as motor evoked potentials (MEPs) and deep brain stimulation local evoked potentials (DLEPs), provide real-time feedback on the physiological effects of the stimulation. By capturing this data, the system 100 can monitor a response of a patient to different stimulation settings, which helps to fine-tune the parameters to achieve the desired therapeutic effects while minimizing side effects.

In step 5215 of method 5200, the system 100 can analyze data using a machine learning model. The data can include the biomarker data as well as historical data. The system 100 can also have access to a database storing the historical data related to previous stimulation parameter selections from the system 100 and physicians. The system 100 can then employ the trained machine learning model to process the data. The system 100 can use the machine learning model to analyze the data to identify patterns and correlations between the stimulation parameters and the patient's physiological responses. This analysis helps in understanding how different settings impact the biomarkers, thereby providing insights into the most effective stimulation parameters. The machine learning model's ability to handle complex, high-dimensional data allows for a more nuanced and accurate analysis (as described above).

In step 5220 of method 5200, the system 100 can generate, in response to analyzing, at least in part, the data, a set of stimulation parameters for deep brain stimulation. Based on the analysis conducted in the step 5215, the system 100 can generate a set of stimulation parameters tailored to the patient's individual needs. The set of stimulation parameters are derived from the machine learning model's predictions, which take into account the recorded biomarker data, historical data and the identified patterns of response. The generated parameters aim to maximize therapeutic benefits while minimizing adverse effects. This step represents the culmination of the system's data-driven approach, providing a scientifically grounded basis for DBS programming that can be further refined through clinical testing (as described above and not repeated herein for brevity).

When generating the set of stimulation parameters for deep brain stimulation (DBS), safety constraints are meticulously considered to ensure patient safety and optimal therapeutic outcomes. The process involves defining safety states and volumes to prevent the system 100 from entering unsafe configurations. Specifically, the system 100 establishes a volume of safety around a desired reference trajectory, ensuring that the system 100 can return to a safe state with high probability. This is achieved by the system 100 encoding the acquisition function into a mixed-integer linear program (MILP), which imposes safety constraints while selecting actions that maximize expected informativeness. The safety constraints ensure that the system 100 can deviate temporarily from the safe region to gain information, provided it can return to a safe state within a specified time frame. This approach guarantees a minimum probability of safety and allows for the selection of even safer actions when possible, thereby balancing the trade-off between safety and informativeness.

The deep brain stimulation device can then be configured to deliver electromagnetic energy to the portions of the brain based, at least in part, on the set of stimulation parameters generated by the system 100 using the machine learning model. In step 5225 of method 5200, the process terminates.

The features and other aspects and principles of the disclosed embodiments may be implemented in various environments. Such environments and related applications may be specifically constructed for performing the various processes and operations of the disclosed embodiments or they may include a general-purpose computer or computing platform selectively activated or reconfigured by program code to provide the necessary functionality. Further, the processes disclosed herein may be implemented by a suitable combination of hardware, software, and/or firmware. For example, the disclosed embodiments may implement general purpose machines configured to execute software programs that perform processes consistent with the disclosed embodiments. Alternatively, the disclosed embodiments may implement a specialized apparatus or system 100 configured to execute software programs that perform processes consistent with the disclosed embodiments. Furthermore, although some disclosed embodiments may be implemented by general purpose machines as computer processing instructions, all or a portion of the functionality of the disclosed embodiments may be implemented instead in dedicated electronics hardware.

The disclosed embodiments also relate to tangible and non-transitory computer readable media that include program instructions or program code that, when executed by one or more processors, perform one or more computer-implemented operations. The program instructions or program code may include specially designed and constructed instructions or code, and/or instructions and code well-known and available to those having ordinary skill in the computer software arts. For example, the disclosed embodiments may execute high level and/or low-level software instructions, such as machine code (e.g., such as that produced by a compiler) and/or high-level code that can be executed by a processor using an interpreter.

The technology disclosed herein typically involves a high-level design effort to construct a computational system 100 that can appropriately process unpredictable data. Mathematical algorithms may be used as building blocks for a framework, however certain implementations of the system 100 may autonomously learn their own operation parameters, achieving better results, higher accuracy, fewer errors, fewer crashes, and greater speed.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology described above with reference to user devices may include mobile computing devices. Those skilled in the art recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

In this description, numerous specific details have been set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features may alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A system for selecting parameters for a deep brain stimulation device comprising:
at least one processor;
a memory in communication with the at least one processor and having stored thereon instructions that, when executed by the at least one processor, is configured to cause the system to:
analyze, using a machine learning model, data from the deep brain stimulation device or an electromyography; and
generate, in response to analyzing, at least in part, the data, a set of stimulation parameters for deep brain stimulation.

2. The system of claim 1, wherein the data comprises, at least in part, sample data from a neural network or biomarker data collected from a population of patients.

3. The system of claim 2, wherein the biomarker data collected from the population of patients was recorded by the deep brain stimulation device or the electromyography.

4. The system of claim 1, wherein the set of stimulation parameters is generated using the machine learning model trained to select parameters using historical data of previous sets of stimulation parameters.

5. The system of claim 1, wherein the machine learning model is configured to maximize deep brain stimulation local evoked potentials (DLEP) and minimize EMG-measured motor evoked potentials (mEP).

6. The system of claim 5, wherein the machine learning model is configured to predict biomarker values for unseen parameters by programming deep brain stimulation parameters in a simulation environment.

7. The system of claim 1, wherein the instructions, when executed by the at least one processor, is configured to cause the system to instruct the deep brain stimulation device to deliver electromagnetic energy to at least a portion of a brain of a user, wherein one or more characteristics of the electromagnetic energy is based, at least in part, on the set of stimulation parameters.

8. The system of claim 1, wherein the memory, when executed by the at least one processor, is further configured to cause the system to:
determine whether at least a portion of the set of stimulation parameters fall within a predetermined safety threshold.

9. The system of claim 1, wherein the machine learning model is configured to:
predict a probability of returning to a safe state;
take an action outside of the safe state to gain information; and
return to the safe state within a preset time period.

10. A method for selecting parameters for a deep brain stimulation device comprising:
analyzing, using a machine learning model, data from the deep brain stimulation device or an electromyography; and
generating, in response to analyzing, at least in part, the data, a set of stimulation parameters for deep brain stimulation.

11. The method of claim 10, wherein the data comprises, at least in part, sample data from a neural network or biomarker data collected from a population of patients.

12. The method of claim 10, wherein the set of stimulation parameters is generated using the machine learning model trained to select parameters using historical data of previous sets of stimulation parameters.

13. The method of claim 10, further comprising:
instructing the deep brain stimulation device to deliver electromagnetic energy to at least a portion of a brain of a user, wherein one or more characteristics of the electromagnetic energy is based, at least in part, on the set of stimulation parameters.

14. The method of claim 13, wherein the machine learning model is configured to predict biomarker values for unseen parameters by programming deep brain stimulation parameters in a simulation environment.

15. The method of claim 13, further comprising:
predicting, using the machine learning model, a probability of returning to a safe state;
taking an action outside of the safe state to gain information; and
returning to the safe state within a preset time period.

16. A non-transitory computer readable medium having stored thereon instructions comprising executable code which when executed by one or more processors, causes the one or more processors to:
analyze, using a machine learning model, data from a deep brain stimulation device or an electromyography; and
generate, in response to analyzing, at least in part, the data, a set of stimulation parameters for deep brain stimulation.

17. The non-transitory computer readable medium of claim 16, wherein the data comprises, at least in part, sample data from a neural network or biomarker data collected from a population of patients.

18. The non-transitory computer readable medium of claim 16, wherein the machine learning model is configured to select the set of stimulation parameters by testing parameters in a simulation environment using historical data of previous sets of stimulation parameters.

19. The non-transitory computer readable medium of claim 16, wherein the machine learning model is configured to predict biomarker values for unseen parameters while maximizing deep brain stimulation local evoked potentials (DLEP) and minimizing EMG-measured motor evoked potentials (mEP).

20. The non-transitory computer readable medium of claim 16, wherein the instructions further comprise executable code which when executed by one or more processors, causes the one or more processors to:
predicting, using the machine learning model, a probability of returning to a safe state;
taking an action outside of the safe state to gain information; and
returning to the safe state within a preset time period.
